(19) United States
(12) Patent Application Publication
(10) Pub. No.: US 2024/0390377 A1
(43) Pub. Date: Nov. 28, 2024

(54) COMPOSITION FOR THE PREVENTION OR TREATMENT OF NEURODEGENERATIVE OR MOTOR NEURON DISEASES COMPRISING HALOFUGINONE AS AN ACTIVE INGREDIENT

(71) Applicants: CURAMYS CO., LTD., Seoul (KR); SEOUL NATIONAL UNIVERSITY HOSPITAL, Seoul (KR)

(72) Inventors: Jung Joon SUNG, Seongnam-si Gyeonggi-do (KR); Young Nam KWON, Seoul (KR); Do Yeon LEE, Seoul (KR)

(21) Appl. No.: 18/775,636

(22) Filed: Jul. 17, 2024

Related U.S. Application Data
(63) Continuation of application No. PCT/KR2022/020102, filed on Dec. 12, 2022.

(30) Foreign Application Priority Data
Jan. 18, 2022 (KR) 10-2022-0007094

(51) Int. Cl. A61K 31/517; A61P 25/14; A61P 25/28

(52) U.S. Cl. CPC A61K 31/517; A61P 25/14; A61P 25/28

(57) ABSTRACT

The present invention relates to a composition for the prevention or treatment of neurodegenerative or motor neuron diseases comprising halofuginone as an active ingredient. Specifically, in Amyotrophic Lateral Sclerosis (ALS) cell and animal models, halofuginone inhibits fibrosis induced by elevated TGF-β, enhances skeletal muscle formation, improves joint contracture, and exhibits dual effects of suppressing inflammatory responses and neuronal cell death in the central nervous system, and then, halofuginone leads to the delay in the progression of ALS symptoms, improvement in performance capabilities, and extension of survival duration. Consequently, halofuginone can be usefully utilized as an active ingredient in a composition for the prevention or treatment of neurodegenerative or motor neuron diseases including ALS.

### CROSS REFERENCE TO RELATED APPLICATIONS

This application is a continuation of international application of PCT/KR2022/020102 filed 12 Dec. 2022, which claims the benefit of, and relies on the filing date of, Korean Patent Application No. 10-2022-0007094 filed 18 Jan. 2022, the entire disclosure of which is incorporated herein by reference.

### SEQUENCE LISTING

The instant application contains a Sequence Listing which has been submitted electronically in XML format and is hereby incorporated by reference in its entirety. Said XML copy, created on 1 Feb. 2024, is named Sequence Listing 0363-0043-PCT-US-B.xml and is 54,353 bytes in size.

### TECHNICAL FIELD

The present invention relates to a composition for the prevention or treatment of neurodegenerative or motor neuron diseases comprising halofuginone as an active ingredient.

### BACKGROUND ART

Amyotrophic lateral sclerosis (ALS), Alzheimer's disease, Parkinson's disease, Huntington's disease, multiple sclerosis and the like are representative degenerative nerve diseases or motor neuron diseases. Currently clinically used treatments are extremely limited.

ALS degrades the quality of life and death due to the progression of muscle atrophy, joint contracture and pain. In addition, the clinical course and prognosis are heterogeneous due to various pathophysiological mechanisms, including genetic mutations, protein homeostasis, mitochondrial dysfunction, neurological dysfunction, and neuroinflammation.

Transforming growth factor-β (TGF-β) is a multifunctional cytokine involved in cell regulation, including cell growth, differentiation, and apoptosis. Following the identification of an association between TGF-β and ALS, the role of TGF-β in ALS progression has begun to be elucidated. In skeletal muscle, TGF-β plays a role in repairing damaged muscles and generally regulates myogenesis, growth, and differentiation. However, sustained elevation of TGF-β leads to reduced myogenesis and promotes muscle fibrosis and atrophy. Previous studies using ALS murine models have demonstrated enhanced TGF-β signaling pathways in symptomatic stages, resulting in abundant production of extracellular matrix (ECM) from increased fibro/adipogenic progenitors. Similarly, in the normal nervous system, it is known to protect neurons from excitotoxicity and oxidative damage and play an important role in neurogenesis. However, it has been reported that TGF-β, which has been continuously elevated, is associated with the promotion of ALS disease progression.

Additionally, it has been reported that the TGF-β signaling pathway is associated with not only ALS but also Alzheimer's disease, Parkinson's disease, Huntington's disease, and multiple sclerosis.

Consequently, as a result of our efforts to develop a therapeutic agent capable of preventing the acceleration of neurodegenerative or motor neuron diseases, including ALS, induced by continuous TGF-β activity and demonstrating therapeutic effects, it was confirmed that in ALS cell and animal models, halofuginone inhibits fibrosis induced by elevated TGF-β, enhances skeletal muscle formation, improves joint contracture, and exhibits dual effects of suppressing inflammatory responses and neuronal cell death in the central nervous system. This leads to the delay in the progression of ALS symptoms, improvement in performance capabilities, and extension of survival duration. By confirming that halofuginone can be usefully utilized as an active ingredient in a composition for the prevention or treatment of neurodegenerative or motor neuron diseases, including ALS, the present application was completed.

### PRIOR ART DOCUMENTS

### Non-Patent Literature

Katsuno, M. et al. Transforming growth factor-β signaling in motor neuron diseases. Current molecular medicine 11, 48-56, 2011.
Kashima, R. & Hata, A. The role of TGF-β superfamily signaling in neurological disorders. Acta biochimica et biophysica Sinica 50, 106-120, 2018.
Peters, S. et al. The TGF-β System As a Potential Pathogenic Player in Disease Modulation of Amyotrophic Lateral Sclerosis. Frontiers in neurology 8, 669, 2017.

### DETAILED DESCRIPTION OF THE INVENTION

### Technical Problem

The object of the present invention is to provide a composition for the prevention or treatment of neurodegenerative or motor neuron diseases, comprising halofuginone or a pharmaceutically acceptable salt thereof as an active ingredient.

### Solution to Problem

To achieve the object of the present invention, the present invention provides a pharmaceutical composition for the prevention or treatment of neurodegenerative or motor neuron diseases, comprising halofuginone or a pharmaceutically acceptable salt thereof as an active ingredient.

The present invention also provides a health functional food composition for the prevention or improvement of neurodegenerative or motor neuron diseases, comprising halofuginone or a pharmaceutically acceptable salt thereof as an active ingredient.

The present invention also provides a method for preventing or treating neurodegenerative or motor neuron diseases, comprising the step of administering to a subject halofuginone or a pharmaceutically acceptable salt thereof.

Furthermore, the present invention provides a pharmaceutical composition comprising halofuginone or a pharmaceutically acceptable salt thereof for use in the prevention or treatment of neurodegenerative or motor neuron diseases.

Moreover, the present invention provides a health functional food composition comprising halofuginone or a pharmaceutically acceptable salt thereof for use in the prevention or improvement of neurodegenerative or motor neuron diseases.

The present invention also provides the use of halofuginone or a pharmaceutically acceptable salt thereof for the manufacture of a medicament for the prevention or treatment of neurodegenerative or motor neuron diseases.

Additionally, the present invention provides the use of halofuginone or a pharmaceutically acceptable salt thereof for the manufacture of a health functional food composition for the prevention or improvement of neurodegenerative or motor neuron diseases.

### Advantageous Effects of Invention

The present inventors have confirmed that in ALS cell and animal models, halofuginone inhibits fibrosis induced by elevated TGF-β, enhances skeletal muscle formation, improves joint contracture, and exhibits dual effects of suppressing inflammatory responses and neuronal cell death in the central nervous system, and then, halofuginone leads to the delay in the progression of ALS symptoms, improvement in performance capabilities, and extension of survival duration. Therefore, halofuginone is useful as an active ingredient in compositions for the prevention or treatment of neurodegenerative or motor neuron diseases, including ALS.

### BRIEF DESCRIPTION OF DRAWINGS

FIG. 1 is a diagram schematically illustrating a method of administering halofuginone to an animal model of amyotrophic lateral sclerosis (ALS) according to an embodiment of the present invention.

FIGS. 2A and 2B illustrate TGF-β1, TGF-β2, TGF-β3, α-SMA, MyoD mRNA expression (FIG. 2A) and TGF-β1, α-SMA, and MyoD protein expression (FIG. 2B) after stimulation of TGF-β1 (Transforming Growth Factor-β1) in myoblast.

FIGS. 3A to 3C illustrate cell viability after treatment with various concentrations of halofuginone (FIG. 3A), cell viability after treatment with TGF-β1 and various concentrations of halofuginone (FIG. 3B), and p-Smad2/Samd2 ratio, TGF-β1, α-SMA, MyoD, and collagen I protein expression after treatment with various concentrations of halofuginone or treatment with TGF-β1 and various concentrations of halofuginone (FIG. 3C).

FIGS. 4A to 4C illustrate mRNA expression of TGF-β1, α-SMA, MyoD collagen I (FIG. 4A), protein expression (FIG. 4B) of TGF-β1, α-SMA, MyoD and collagen I, after treatment with TGF-β1 and halofuginone in myoblasts and α-SMA and MyoD expression (FIG. 4C) confirmed by immunocytochemical analysis.

FIGS. 5A and 5B illustrate the mRNA expression of TGF-β1, α-SMA, and collagen I (FIG. 5A) and the p-Smad2/Smad2 ratio, TGF-β1, α-SMA, and collagen I protein expression (FIG. 5B) after treatment with halofuginone in fibroblasts isolated from an ALS animal model.

FIGS. 6A to 6C illustrate the changes in motor function and body weight (FIG. 6A), and the changes in symptom onset (FIGS. 6B and 6C) after administration of halofuginone to an ALS animal model.

FIGS. 7A to 7D illustrate immunohistochemical analysis of TGF-β1, α-SMA, MyoD, and collagen I expression in 90-day-old females (FIG. 7A) and males (FIG. 7B), 120-day-old females (FIG. 7C), and males (FIG. 7D) after administering halofuginone to ALS animal models.

FIGS. 8A and 8B illustrate the range of motion (ROM) in the knee joint synovial cavity (FIG. 8A) and p-Smad2/Samd2 ratio, TGF-β1, α-SMA, MyoD, and collagen I protein expression (FIG. 8B) at 120 days of age, after administering halofuginone to ALS animal models.

FIGS. 9A to 9F illustrate microglia activity (FIG. 9A), astrocyte activity (FIG. 9B), IL-1β expression (FIG. 9C), the number of ChAT-positive motor neurons (FIG. 9D), ChAT mRNA expression (FIG. 9E) and ChAT protein expression (FIG. 9F) in the lumbar spine at 120 days of age, after administering halofuginone to ALS animal models.

FIGS. 10A and 10B illustrate microglia activity (FIG. 10A), and the number of ChAT-positive motor neurons (FIG. 10B) in the lumbar spine at 90 days of age, after administering halofuginone to ALS animal models.

FIGS. 11A and 11B illustrate TGF-β, iNOS, CD86, arginase 1, IFN-α, IL-6, IL-1β, TNF-α, caspase-3, bcl2, bac mRNA expression (FIG. 11A), and cleaved caspase-3, bcl2, and bax protein expression (FIG. 11B) in the lumbar spine at 120 days of age, after administering halofuginone to ALS animal models.

### BEST MODE FOR CARRYING OUT THE INVENTION

Hereinafter, the present invention will be described in more detail.

In the present invention, the term “prevention” refers to all actions that inhibit or delay the onset, acceleration, spread, or recurrence of a disease by administering the composition of the present invention. The term “treatment” refers to all actions that improve or beneficially alter the symptoms of the disease by administering the composition of the present invention.

In the present invention, the term “administration” means providing a predetermined substance to a patient by any suitable method. The administration route of the composition of the present invention can be oral or parenteral through any general route, as long as it can reach the target tissue. Furthermore, the composition can be administered by any device that allows the active substance to move to the target cells.

The present invention provides a pharmaceutical composition for the prevention or treatment of neurodegenerative or motor neuron diseases, comprising halofuginone or a pharmaceutically acceptable salt thereof as an active ingredient.

Additionally, the present invention provides a method for the prevention or treatment of neurodegenerative or motor neuron diseases, comprising administering halofuginone or a pharmaceutically acceptable salt thereof to a subject.

Furthermore, the present invention provides a pharmaceutical composition comprising halofuginone or a pharmaceutically acceptable salt thereof for use in the prevention or treatment of neurodegenerative or motor neuron diseases.

The present invention also provides the use of halofuginone or a pharmaceutically acceptable salt thereof for the manufacture of a medicament for the prevention or treatment of neurodegenerative or motor neuron diseases.

In the present invention, halofuginone or its pharmaceutically acceptable salt, as a TGF-β inhibitor, has the chemical structure represented by the following [Chemical Formula 1]:

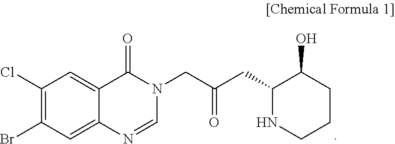 [SMILES: O=C(C[C@H]1NCCC[C@@H]1O)Cn1cnc2cc(Br)c(Cl)cc2c1=O]

Additionally, halofuginone may be used either commercially available or synthesized.

In the present invention, the neurodegenerative or motor neuron diseases may include, but are not limited to, Amyotrophic Lateral Sclerosis, Alzheimer's disease, Parkinson's disease, Huntington's disease, multiple sclerosis, dystonia, spinal muscular atrophy, or inflammatory neuropathy.

Specifically, in neurodegenerative or motor neuron diseases, halofuginone can alleviate fibrosis of the joint synovial cavity, induced by TGF-β stimulation and enhance skeletal muscle formation, thereby preventing or treating the diseases.

Furthermore, in neurodegenerative or motor neuron diseases, halofuginone can inhibit inflammatory responses and neuronal cell death in the central nervous system induced by TGF-β stimulation, thereby preventing or treating the diseases. More specifically, halofuginone can increase the expression of anti-inflammatory factors such as arginase 1 in the central nervous system and decrease the expression of pro-inflammatory factors such as iNOS, CD86, IFN-α, TNF-α, IL-1β, or IL-6, to prevent or treat the diseases. Additionally, halofuginone can increase the expression of anti-neuronal cell death factors such as bcl-2 and decrease the expression of neuronal cell death factors such as caspase-3 and bax in the central nervous system, to prevent or treat the diseases.

Furthermore, in neurodegenerative or motor neuron diseases, halofuginone can delay the progression of symptoms, improves performance capabilities and extends survival duration.

In a specific embodiment of the present invention, the inventors of the present invention confirmed that while fibrosis was induced and myogenesis was impaired in myoblasts stimulated with TGF-β1, treatment with halofuginone inhibited the induction of fibrosis and the impairment of myogenesis induced by TGF-βl.

Furthermore, the inventors of the present invention confirmed that treatment with halofuginone inhibited fibrosis and decreased muscle transcription factors in fibroblasts isolated from an animal model of Amyotrophic Lateral Sclerosis (ALS).

Furthermore, the inventors of the present invention confirmed that treatment with Halofuginone inhibited fibrosis and decreased muscle transcription factors in fibroblasts isolated from the Amyotrophic Lateral Sclerosis (ALS) animal model.

The inventors of the present invention also confirmed that administration of halofuginone in an ALS animal model resulted in delayed onset of ALS, improved performance capabilities, and extended survival duration. Additionally, it was observed that administration of halofuginone in the ALS animal model led to alleviated fibrosis of the joint synovial cavity, enhanced skeletal muscle formation, and improved joint contracture. Moreover, halofuginone administration in the ALS animal model demonstrated anti-inflammatory and neuronal cell death inhibitory effects in the central nervous system (CNS).

Therefore, the inventors of the present invention confirmed that in ALS cell and animal models, halofuginone inhibits fibrosis, enhances skeletal muscle formation, improves joint contracture, and exhibits dual effects of suppressing inflammatory responses and neuronal cell death in the central nervous system, and then, halofuginone leads to the delay in the progression of ALS symptoms, improvement in performance capabilities, and extension of survival duration. Consequently, halofuginone can be usefully utilized as an active ingredient in a composition for the prevention or treatment of neurodegenerative or motor neuron diseases including ALS.

The halofuginone of the present invention encompasses all pharmaceutically acceptable salts and possible solvates, hydrates, racemates, or stereoisomers that can be prepared therefrom.

The halofuginone of the present invention can be used in the form of a pharmaceutically acceptable salt, and an acid addition salt formed by a pharmaceutically acceptable free acid is useful as the salt. Acid addition salts are obtained from inorganic acids such as hydrochloric, nitric, phosphoric, sulfuric, hydrobromic, hydroiodic, nitrous, and phosphorous acids, or from non-toxic organic acids such as aliphatic mono and dicarboxylates, phenyl-substituted alkanoates, hydroxy alkanoates and alkandioates, aromatic acids, aliphatic and aromatic sulfonic acids. Such pharmacologically innocuous salts include sulfates, pyrosulfates, bisulfates, sulfites, bisulfites, nitrates, phosphates, monohydrogen phosphates, dihydrogen phosphates, metaphosphates, pyrophosphates, chlorides, bromides, iodides, fluorides, acetates, propionates, decanoates, caprylates, acrylates, formates, isobutyrates, caprates, heptanoates, propiolates, oxalates, malonates, succinates, suberates, sebacates, fumarates, maleates, butane-1,4-dioates, hexane-1,6-dioates, benzoates, chlorobenzoates, methylbenzoates, dinitrobenzoates, hydroxybenzoates, methoxybenzoates, phthalates, terephthalates, benzene sulfonates, toluene sulfonates, chlorobenzene sulfonates, xylene sulfonates, phenyl acetates, phenyl propionates, phenyl butyrates, citrates, lactates, hydroxybutyrates, glycolates, maleates, tartrates, methanesulfonates, propanesulfonates, naphthalene-1-sulfonates, naphthalene-2-sulfonates, or mandelates.

The acid addition salt according to the present invention can be prepared by conventional methods, for example, by dissolving the compound of the present invention in an excess of an acid aqueous solution and precipitating the salt using a water-mixed organic solvent such as methanol, ethanol, acetone or acetonitrile. Alternatively, it may be prepared by evaporating a solvent or an excess acid from the mixture to dry the mixture or by suction filtration of the precipitated salt.

Additionally, pharmaceutically acceptable metal salts can be made using a base. Alkali metal or alkaline earth metal salts can be prepared, for example, by dissolving the compound in an excess of an alkali metal hydroxide or alkaline earth metal hydroxide solution, filtering the insoluble compound salts, and evaporating and drying the filtrate. In this case, it is pharmaceutically suitable to prepare metal salts such as sodium, potassium, or calcium salts. Additionally, the corresponding silver salt can be obtained by reacting the alkali metal or alkaline earth metal salt with an appropriate silver salt (e.g., silver nitrate).

Halofuginone or a pharmaceutically acceptable salt thereof of the present invention may be included in a therapeutic amount with a pharmaceutically acceptable carrier.

The term “effective amount” as used herein means the amount of the compound of the present invention sufficient to delay or minimize neurodegenerative and/or motor neuron disease; or to provide therapeutic benefits in the treatment or management of neurodegenerative and/or motor neuron disease.

The pharmaceutically acceptable carriers can include, for example, carriers for oral or parenteral administration. Carriers for oral administration may include lactose, starch, cellulose derivatives, magnesium stearate, stearic acid, and the like. Additionally, carriers for parenteral administration may include water, suitable oils, saline, aqueous glucose, glycols, and the like and may further include stabilizers and preservatives. Suitable stabilizers include antioxidants such as sodium bisulfite, sodium sulfite, or ascorbic acid. Suitable preservatives include benzalkonium chloride, methyl- or propyl-paraben, and chlorobutanol. Other pharmaceutically acceptable carriers can be referred to in the following literature. (Remington's Pharmaceutical Sciences, 19th ed., Mack Publishing Company, Easton, PA, 1995).

The pharmaceutical composition of the present invention can be administered by any route to mammals, including humans. It can be administered orally or parenterally.

The parenteral administration method may be, for example, intravenous, intramuscular, arterial, intracerebral, intrathecal, intracardiac, percutaneous, subcutaneous, intraperitoneal, intranasal, intestinal, topical, sublingual, or rectal administration, but is not limited thereto. For example, the pharmaceutical composition of the present invention may be prepared in an injection-type formulation, and may be administered by a method of lightly pricking the skin with a thin 30-gauge injection needle, or by directly applying or adhering the composition to the skin.

The pharmaceutical composition of the present invention can be formulated into formulations for oral administration or parenteral administration according to the administration route as described above.

In the case of oral administration formulations, the composition of the present invention may be formulated using methods known in the art, such as powder, granule, tablet, pills, sugar tablet, capsule, liquid, gel, syrup, slurry, suspension, etc. For example, oral formulations can obtain tablets by blending the active ingredients with solid excipients, then crushing them, adding suitable supplements, and processing them into a granular mixture. Examples of suitable excipients may include sugars including lactose, dextrose, sucrose, sorbitol, mannitol, xylitol, erythritol and maltitol, starch including corn starch, wheat starch, rice starch and potato starch, cellulose including cellulose, methyl cellulose, sodium carboxymethylcellulose and hydroxypropylmethyl-cellulose, and fillers such as gelatin and polyvinylpyrrolidone. Also, in some cases, crosslinked polyvinylpyrrolidone, agar, alginic acid, sodium alginate, or the like may be added as a disintegrant. Furthermore, the pharmaceutical composition of the present invention may further include an anticoagulant, a lubricant, a wetting agent, a flavoring agent, an emulsifier, and a preservative.

In the case of parenteral administration formulations, they can be formulated by methods known in the art in the form of injections, creams, lotions, external ointments, oils, moisturizers, gels, patches, aerosols, and nasal aspirants. These formulations are described in the prescription literature commonly known in all pharmaceutical chemistry (Remington's Pharmaceutical Science, 15th Edition, 1975. Mack Publishing Company, Easton, Pennsylvania 18042, Chapter 87: Blaug, Seymour).

The total dose of the pharmaceutical composition of the present invention can be administered to a patient as a single dose, more specifically as a single dose administered over an extended period, or it can be administered over an extended period as multiple doses through a fractionated treatment protocol. The pharmaceutical composition of the present invention can vary the amount of active ingredients according to the symptoms of the disease. Specifically, the total dosage of the composition of the present invention may range from 0.01 μg to 1,000 mg per 1 kg of patient body weight per day, more specifically from 0.1 μg to 100 mg. However, the dose of the pharmaceutical composition of the present invention will allow those skilled in the art to determine an appropriate effective dose, taking into account various factors such as the patient's age, weight, health status, gender, disease severity, diet, and excretion rate, as well as the route and number of treatments. The pharmaceutical composition according to the present invention is not specifically limited to any particular formulation, route of administration, or method of administration as long as it exhibits the effects of the present invention.

Furthermore, the pharmaceutical composition of the present invention can be administered as a monotherapy or in combination with other therapeutic agents. When administered in combination with other therapeutic agents, the composition of the present invention and the other therapeutic agents can be administered concurrently, individually, or sequentially. In this case, the other therapeutic agent can be a substance already known to have treatment or ameliorating effects of neurodegenerative and/or motor neuron disease. When the pharmaceutical composition of the present invention is administered in combination with other therapeutic agents, the composition of the present invention and other therapeutic agents can be separately formulated in separate containers, or can be complex formulated together in the same formulation.

The present invention also provides a health functional food composition for the prevention or improvement of neurodegenerative or motor neuron diseases, comprising halofuginone or a pharmaceutically acceptable salt thereof as an active ingredient.

Additionally, the present invention provides a health functional food composition comprising halofuginone or a pharmaceutically acceptable salt thereof for use in the prevention or improvement of neurodegenerative or motor neuron diseases.

Furthermore, the present invention provides the use of halofuginone or a pharmaceutically acceptable salt thereof for the manufacture of a health functional food composition for the prevention or improvement of neurodegenerative or motor neuron diseases.

In the present invention, the contents of the above halofuginone or pharmaceutically acceptable salt thereof and neurodegenerative or motor neuron diseases are the same as described above, so a detailed explanation will refer to the above and only the specific composition of the health functional food composition will be described below.

The present inventors have confirmed that in ALS cell and animal models, halofuginone inhibits fibrosis induced by elevated TGF-β, enhances skeletal muscle formation, improves joint contracture, and exhibits dual effects of suppressing inflammatory responses and neuronal cell death in the central nervous system, and then, halofuginone leads to the delay in the progression of ALS symptoms, improvement in performance capabilities, and extension of survival duration. Therefore, the aforementioned halofuginone is useful as an active ingredient in health functional food compositions for the prevention or improvement of neurodegenerative or motor neuron diseases, including ALS.

The health functional food composition of the present invention can be manufactured in any one of the formulations selected from the group consisting of powder, granule, pill, tablet, capsule, candy, syrup, and beverage, but is not limited thereto.

The health functional food composition is not particularly limited as long as it can be ingested to prevent or improve neurodegenerative or motor neuron diseases. When using the health functional food composition of the present invention as a food additive, it can be added as is or used together with other foods or food ingredients and can be appropriately used according to conventional methods. The active ingredient can be appropriately used according to its intended use (prevention or improvement). Generally, when manufacturing food or beverages, it is added in an amount of 15 parts by weight or less, preferably 10 parts by weight or less, based on the health functional food composition of the present invention. However, in the case of long-term consumption for health regulation purposes, the amount may be less than the aforementioned range, and since there is no safety issue, the active ingredient can be used in an amount exceeding the aforementioned range. There is no particular limitation on the type of food. Examples of foods to which the health functional food composition can be added include meat, sausage, bread, chocolate, candies, snacks, cookies, pizza, ramen, other noodles, gum, dairy products including ice cream, various soups, beverages, tea drinks, alcoholic drinks, and vitamin complexes, as well as all health foods in the conventional sense. Additionally, the health functional food composition of the present invention can be manufactured as food, particularly functional food.

The functional food of the present invention includes components that are conventionally added during food manufacturing, such as proteins, carbohydrates, fats, nutrients, and seasonings. For example, when manufactured as a drink, it may include natural carbohydrates or flavoring agents as additional components, in addition to the active ingredient. The natural carbohydrates are preferably monosaccharides (e.g., glucose, fructose), disaccharides (e.g., maltose, sucrose), oligosaccharides, polysaccharides (e.g., dextrin, cyclodextrin), or sugar alcohols (e.g., xylitol, sorbitol, erythritol). The flavoring agents can be natural flavoring agents (e.g., thaumatin, stevia extract) or synthetic flavoring agents (e.g., saccharin, aspartame). In addition to the health functional food composition mentioned above, various nutrients, vitamins, electrolytes, flavor enhancers, colorants, pectic acid and its salts, alginic acid and its salts, organic acids, protective colloidal thickners, viscosity modifiers, pH regulators, stabilizers, preservatives, glycerin, alcohols, carbonating agents used in carbonated beverages and the like may also be included.

Hereinafter, the present invention will be described in detail with reference to examples and experimental examples.

However, the following examples and experimental examples are provided for illustrative purposes only, and the scope of the present invention is not limited to these examples.

### <Example 1> Culturing of Myoblast Cells and Treatment with TGF-β1 (Transforming Growth Factor-1) and Halofuginone

To investigate the effects of TGF-β (Transforming growth factor-β) on myoblast cells and the effects of halofuginone, mouse myoblast cells were cultured and treated with TGF-β1 and halofuginone.

Specifically, C2C12 cells (CRL-1772) were purchased from the American Type Culture Collection (ATCC) and maintained in DMEM medium (Welgene) containing 10% (v/v) fetal bovine serum (FBS; Gibco) and 1% penicillin-streptomycin (P/S; Gibco) at 37° C. under 5% CO₂ conditions. The C2C12 cells were seeded in 6-well plates at a density of 3×10⁵ cells per well and allowed to adhere for 24 hours. Subsequently, the medium was replaced with serum-free medium after 24 hours, followed by stimulation with 5 ng/ml rhTGF-β1 (recombinant human TGF-β1; R&D Systems). After stimulation, the cells were treated with various concentrations (0 [control], or 1, 2.5, 5 [low dose], 10 [high dose], 20, 50, 100 ng/ml) of halofuginone (Sigma) in serum-free medium for 24 hours.

### <Example 2> Cell Viability Analysis

The cell viability of C2C12 cells was measured using the Cell Counting Kit-8 (CCK-8 kit) analysis (Enzo Life Sciences, ALX-850-039) under serum-free conditions after treatment with rhTGF-β1 or halofuginone.

Specifically, the C2C12 cells from <Example 1> were cultured in a 96-well plate (10⁴ cells/well) for 24 hours, followed by stimulation with 5 ng/ml rhTGF-β1 for 24 hours. After stimulation, halofuginone was treated at various concentrations (0 [control], or 1, 2.5, 5, 10, 20, 50, 100 ng/ml) for 24 hours. After 24 hours, 10 μl of CCK-8 solution was added to each well and incubated at 37° C. for 1 hour. Subsequently, absorbance at 450 nm was measured using the VersaMax microplate reader (Molecular Devices). The cell viability was expressed as the percentage relative to the viability of the control group (untreated cells). The average absorbance values obtained from six wells for each concentration of halofuginone were calculated.

### <Example 3> Immunocytochemical Analysis

Immunocytochemical analysis was performed on C2C12 cells under serum-free conditions after treatment with rhTGF-β1 or halofuginone, as described in <Example 1>.

Specifically, C2C12 cells were plated in 6-well plates with coverslips and stimulated with rhTGF-β1 in serum-free medium. After 24 hours of stimulation, the cells were treated with low or high concentrations of halofuginone in serum-free medium for 24 hours. The cells were washed three times with PBS and fixed in 4% paraformaldehyde at room temperature for 15 minutes. The fixed cells were then washed three times with PBS and treated with 0.5% Triton X-100 for 5 minutes. Subsequently, they were blocked with 5% bovine serum albumin in 0.3% PBS-T for 1 hour at room temperature. The cells were incubated with anti-α-SMA antibody (1:200; abcam cat #ab7817) and anti-MyoD antibody (1:200; Santa Cruz, cat #sc377460) in blocking buffer at 4° C. for 24 hours. After washing three times with PBS, the cells were incubated with Alexa Fluor secondary antibodies at room temperature for 1 hour. Nuclear DNA was stained with DAPI (4,6-diamino-2-phenylindole, 1:1,000; Sigma), washed twice with PBS, and coverslips were mounted on glass slides using a Dako fluorescence mounting medium (Dako). Images were observed with a Leica TCS SP8 confocal microscope (Leica Microsystems, Wetzlar, Germany) using a 40× objective lens. In addition, it was quantified in a blind manner using Leica Application Suite X software (Leica Microsystems).

### <Example 4> Genetic Analysis of Amyotrophic Lateral Sclerosis (ALS) Animal Model

To investigate the efficacy of halofuginone in amyotrophic lateral sclerosis (ALS), the ALS animal model was used.

Specifically, all animal experiments were conducted according to the guidelines of the Animal Care Committee of Seoul National University Animal Hospital (IACUC, approval number SNU-200220-1-2). Transgenic mice expressing the human G93A mutant SOD1 gene (B6SJL-Tg(SOD1-G93A) 1 Gur/J) as an ALS animal model were purchased from Jackson Laboratory (Bar Harbor, ME, USA). Male transgenic ALS mice expressing mtSOD1(G93A) H1 strain (Jackson Laboratories, Bar Harbor, ME, USA) were crossed with female mice (B6/SJLF1). The mice were housed under standard conditions with a constant temperature of 22±1° C., relative humidity of 40%, and a 12-hour light-dark cycle, with ad libitum access to food and water. Genotypes were confirmed by polymerase chain reaction (PCR) using primers shown in [Table 1] below, and the number of transgene copies was confirmed. Next, G93A mutant SOD1 transgenic mice were divided into three stages: 60 days old (asymptomatic), 90 days old (early symptom), and 120 days old (late symptom) depending on the time point. A total of 96 mice were used in the experiment. As a control group, non-transgenic mice were used.

TABLE 1

| Primer | | Sequence (5′-3′) | Sequence number |
|---|---|---|---|
| hSOD1wt | Forward | CTAGGCCACAGAATTGAAAGATCT | 1 |
| | Reverse | GTAGGTGGAAATTCTAGCATCATCC | 2 |
| hSOD1G93A | Forward | CATCAGCCCTAATCCATCTGA | 3 |
| | Reverse | CGCGACTAACAATCAAAGTGA | 4 |

### <Example 5> Culture of ALS-Derived Fibroblasts and Treatment with Halofuginone

To investigate the efficacy of halofuginone in ALS, fibroblasts were isolated from an ALS animal model in which the G93A mutant SOD1 gene was identified in <Example 4>, and the cells were treated with halofuginone.

Specifically, primary fibroblasts were isolated from skeletal muscle of 120-day-old non-transformed or G93A mutant SOD1 transgenic mice of Example 4. The excised tissue was placed on 100-mm culture dishes containing HBSS (Hanks' Balanced Salt Solution; Gibco, cat #14175095) and penicillin-streptomycin (Cat #15070-063, Thermo Fisher Scientific) without calcium and magnesium. Muscle tissue was cut on ice blocks. Then, the tissue was cut into 1 mm pieces and enzymatically reacted with 0.2% IV type collagenase (Sigma) at 37° C. for 1 hour. The enzymatic reaction was stopped by the addition of 10 ml of FBS (Gibco). The tissue mixture was centrifuged at 1,800 rpm at 4° C. for 5 min and resuspended with DMEM medium (Welgene) with 10% FBS. Cells were filtered through a 70 μm cell filter (BD Biosciences, San Jose, CA) and incubated in 0.2% gelatin-coated plates with DMEM medium with 10% FBS, 1% penicillin-streptomycin. Then, halofuginone was treated in the same manner as described in Example 1.

### <Example 6> Administration of Halofuginone in ALS Animal Model

To investigate the efficacy of halofuginone in ALS, halofuginone was administered to the ALS animal model in which the G93A mutant SOD1 gene was identified in <Example 4>.

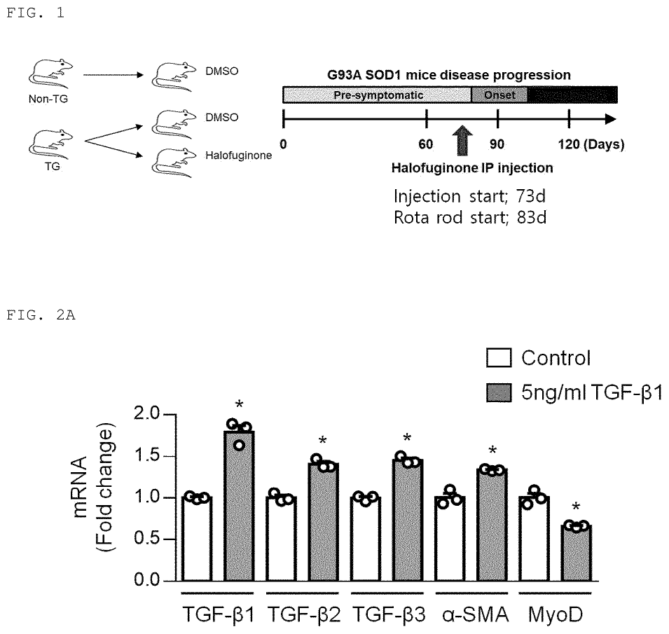

Specifically, as depicted in FIG. 1, the mice matching the sexes and ages of Example 4 were randomly divided into three groups, and as shown in Table 2 below, G93A mutant SOD1 transgenic mice of the DMSO TG group and the Hal TG group were injected intraperitoneally (i.p.) with vehicle (DMSO, Duchefa Biochemie, D1370) and halofuginone (Sigma, S8144) three times per week for over 10 weeks starting at 10 weeks of age (early symptom stage). As a control group, non-transgenic mice were injected intraperitoneally (i.p.) with vehicle (DMSO) in the same manner for the same period of time.

TABLE 2

| | | Number | Sample | | |
|---|---|---|---|---|---|
| | | of | Administered | | Administration |
| Group | Gender | animals | sample | Dosage | method |
| DMSO | Male | 16 | Vehicle(DMSO) | 5 | intraper- |
| Non-TG | Female | 16 | | [symbol] / | itoneally, |
| DMSO | Male | 16 | | mouse | three times |
| TG | Female | 16 | | | a week |
| Hal | Male | 16 | Halofuginone | 250 | |
| TG | Female | 16 | | μg/kg | |

Meanwhile, G93A mutant SOD1 transgenic mice or non-transgenic mice used in the biochemical assay were sacrificed at 90 and 120 days of age, and all experiments were performed in triplicate.

### <Example 7> ALS Animal Model Disease Progression, Survival, and Motor Function Analysis

To investigate the efficacy of halofuginone in ALS, behavioral experiments were performed in a single blind manner after administration of halofuginone to the ALS animal model as in Example 6.

Specifically, the clinical condition and body weight of the mice in Example 6 were assessed three times a week starting from day 83 post-birth. Disease onset was defined as tremor of the limbs when the mouse's tail was suspended in the air, reported to be clinically associated with upper motor neuron system [S. Nagano, Y. Fujii, T. Yamamoto, M. Taniyama, K. Fukada, T. Yanagihara, et al. The efficacy of trientine or ascorbate alone compared to that of the combined treatment with these two agents in familial amyotrophic lateral sclerosis model mice Exp Neurol, 179 (2003), pp. 176-180]. The endpoint age was determined as the inability of the animal to right itself within 30 seconds after being placed on a flat surface on its side. At this point, the mouse was considered deceased [C. Zheng, I. Nennesmo, B. Fadeel, J. I. Henter Vascular endothelial growth factor prolongs survival in a transgenic mouse model of ALS Ann Neurol, 56 (2004), pp. 564-567]. The motor function of mice was assessed using a rotarod apparatus (JD-A-07M, JEUNG DO BIO & PLANT CO., LTD). Mice were trained for one week prior to recording. The mice were placed on a rotarod that started at a speed of 4 rpm and accelerated up to 40 rpm over 300 seconds, increasing by 1 rpm approximately every 8.3 seconds. The rotarod test was measured on average three times per measurement, three times per week from 83 days of age, until it was not possible to stay in rotarod for more than 10 seconds three consecutive times.

### <Example 8> Immunohistochemical Analysis

To investigate the efficacy of halofuginone in ALS, halofuginone was administered to ALS animal models as in Example 6, followed by immunohistochemical analysis using lumbar spinal cords and knee joint tissues.

Specifically, the mice of Example 6 above were perfused with 4% paraformaldehyde (PFA) on 90 and 120 days of age, and the lumbar spinal cord and knee joints were separated. After fixation in 4% PFA for 24 hours, the knee joints were rinsed in running tap water for 24 hours and then incubated at 4° C. with continuous shaking in decalcifying solution (Calci-Clear, HS-104, National Diagnostics). The decalcification solution was replaced daily with fresh solution until the bones were easily penetrated with a needle, indicating completion of the decalcification process. Subsequently, the decalcified knee joints were rinsed in running tap water for 24 hours, and the spinal cord and knee joint samples were treated with 30% sucrose until they sank, followed by freeze protection, followed by successive cuts (14 μm). The lumbar spinal cord and knee joint tissue sections were washed three times with PBS and permeabilized with 0.5% Triton X-100 for 5 minutes. Subsequently, they were blocked with 5% bovine serum albumin (BSA) in 0.3% PBS-T at room temperature for 1 hour. The knee joint tissue sections were incubated with anti-TGF-β1 antibody (1:200; Santa Cruz, cat #sc130348), anti-α-SMA antibody (1:200; Abcam, cat #ab7817), anti-MyoD antibody (1:200; Santa Cruz, cat #sc377460), and anti-Collagen I antibody (1:200; Abcam, cat #ab21286) each, while the lumbar spinal cord sections were incubated with anti-GFAP antibody (1:200; Dako, cat #Z0334), anti-Iba1 antibody (1:200; Wako, cat #016)-20001), anti-TGF-β1 antibody (1:200; Santa Cruz, cat #sc130348), anti-TGF-β1 antibody (1:200; Abcam, cat #ab92486), and anti-IL-1β antibody (1:200; R&D Systems, cat #AF-401-NA) at 4° C. for an additional 24 hours. After washing the sections three times with PBS, they were incubated with Alexa Fluor secondary antibodies in a dark chamber at room temperature for 1 hour. Stained and washed spinal cord sections were stained with DAPI (DAPI; 1:1,000; sigma) for 10 minutes at room temperature, then rinsed twice with PBS, and coverslips were mounted with a Dako fluorescent mounting medium (Dako), and the knee joint was observed with a Leica TCS SP8 confocal microscope (Leica Microsystems, Wetzlar, Germany) using a 7.5× objective lens and the spinal cord was observed with a 9.75× objective lens. Furthermore, analysis was performed using Leica Application Suite X software (Leica Microsystems).

### <Example 9> Motor Neuron Cell Count in Lumbar Spinal Cord

To investigate the efficacy of halofuginone in ALS, halofuginone was administered to ALS animal models as in Example 6, followed by isolation of lumbar spinal cords to count motor neuron cells.

Specifically, mice were sacrificed at various time points (90 days and 120 days) according to the clinical state of the mice of Example 6. Each mouse was transdermally perfused with cold 4% paraformaldehyde (PFA) following cold PBS, and lumbar spinal cord was separated. Samples were post-fixed in 4% paraformaldehyde, treated in a cold 30% sucrose solution, then freeze-protected, and then cross-sectional sections 14 μm thick were successively obtained. Tissue sections were washed with PBS and immersed in PBS containing 0.3% hydrogen peroxide (H₂O₂) for 15 min to remove endogenous peroxidase activity. Sections were washed with PBS, permeabilized with 0.5% Triton X-100 for 5 min, and blocked with 5% bovine serum albumin in 0.3% PBS-T for 1 h at room temperature. Tissue sections from each set were incubated for 24 h with anti-ChAT antibodies (1:400; Millipore, cat #AB144P) as primary antibodies. After washing with PBS, sections were incubated with biotinylated horse anti-goat IgG (H+L) antibody (1:200; Vector Laboratories, cat #BA-9500) for 1 h, washed with PBS, and incubated with peroxidase-conjugated avidin-biotin complex (ABC kit; 1:200; Vector Laboratories, cat #PK4000) for 1 h at room temperature. After thorough washing, sections were incubated in diaminobenzidine (DAB, ImmpACT® DAB Vector Laboratories, cat #SK-4105) to visualize peroxidase staining. Sections were dehydrated and air-dried and mounted with toluene-soluble Permount mounting medium (Fisher Scientific, cat #SP15-500). Motor neurons were quantified from ChAT-stained sections using a computer-assisted microscope (Olympus BX53) at 4× magnification, along with cellSens software. Counts were performed per ventral horn in a total of 3 mice per group. The fields analyzed were all ChAT+ profiles located in the ventral half of the immunostaining section clearly marked on the gray matter of each hemisphere.

### <Example 10> Measurement of Range of Motion (ROM)

To investigate the efficacy of halofuginone in ALS, halofuginone was administered to ALS animal models, as in Example 6, followed by measurement of Range of Motion (ROM) in the knee joints.

Specifically, anatomical indicators were used to determine the knee extension angle. Manual knee joint ROM was measured at the end of disease age (120 days) using Random Two-line, a 2D angle analysis system. The mice of Example 6 were anesthetized with 1% isoflurane inhalation, followed by placing them on an acrylic plate and shaving the skin on their hind legs. The femur was fixed to the plate and a constant force extension moment was applied to the knee joint per mouse. Subsequently, markers were attached to greater trochanter, epicondyle of the knee, and lateral malleolus to obtain markers on the imaging. The angle between the axis of the femur (the greater trochanter to the lateral joint space of the knee) and the fibula (the lateral joint space of the knee to the lateral malleolus) was measured as the knee extension ROM. Sixteen mice in each group were analyzed, and the knees of non-transgenic mice injected with DMSO in the group on day 120 were used as controls.

### <Example 11> Real-Time qRT-PCR Analysis

Real-time qRT-PCR analysis was performed using myoblasts treated with halofuginone after stimulation with TGF-β1 in <Example 1>, fibroblasts treated with halofuginone separated from ALS animal models in <Example 5>, or lumbar spinal cord separated after administration of halofuginone to ALS animal models as in <Example 6>.

Specifically, total RNA was isolated from C2C12 cells in Example 1, fibroblasts in Example 5, and the lumbar spinal cord of mice in Example 6 using the FavorPrep™ Tri-RNA Reagent (Favorgen). The lumbar spinal cord was isolated from the mouse in Example 6 and stored frozen at −80° C. until the experiment. The concentration of total RNA was measured at 260 nm absorbance using a spectrophotometer (NanoDrop Spectrophotometer ND-2000, Thermo Scientific). cDNA was synthesized from 1 μg of total RNA using the ReverTra Ace-α™ (Toyobo) according to the manufacturers protocol. Quantitative RT-PCR was performed using the SYBR Green ExcelTaq™ 2× Fast Q-PCR Master Mix (TQ1200, Smobio) on a thermocycler (CFX Connect Real-Time PCR Detection System, BIO-RAD) with the primers listed in Table 3 below. Fluorescence data were analyzed with Bio-Rad CFX Manager 3.1 software, and control versus relative mRNA expression was calculated by the 2AT) method. Experiments were performed four times on all samples. All primers were designed using Primer3 online software, and GAPDH was used as a control group.

TABLE 3

| Primer | | Seguence (5′-3′) | Sequence Number |
|---|---|---|---|
| GAPDH | Forward | TGTGTCCGTCGTGGATCTGA | 5 |
| GAPDH | Reverse | CCTGCTTCACCACCTTCTTGA | 6 |
| TGF-ß | Forward | CACCGGAGAGCCCTGGATA | 7 |
| TGF-ß | Reverse | TGTACAGCTGCCGCACACA | 8 |
| TGF-ß1 | Forward | TCGCTTTGTACAACAGCACC | 9 |
| TGF-ß1 | Reverse | ACTGCTTCCCGAATGTCTGA | 10 |
| TGF-ß2 | Forward | AGGCAGAGTTCAGGGTCTTC | 11 |
| TGF-ß2 | Reverse | CCTTGCTATCGATGTAGCGC | 12 |
| TGF-ß3 | Forward | AGGAGACCTCGGAGTCTGAG | 13 |
| TGF-ß3 | Reverse | CACTGAGGACACATTGAAACGA | 14 |
| α-SMA | Forward | AGTTTTGTGCTGAGGTCCCTATATG | 15 |
| α-SMA | Reverse | TTCCCAAACAAGGAGCAAAGA | 16 |
| MyoD | Forward | TACAGTGGCGACTCAGATGC | 17 |
| MyoD | Reverse | GAGATGCGCTCCACTATGCT | 18 |
| Collagen I | Forward | CTGGCGGTTCAGGTCCAAT | 19 |
| Collagen I | Reverse | TTCCAGGCAATCCACGAGC | 20 |
| ChAT | Forward | CTGTGCCCCCTTCTAGAGC | 21 |
| ChAT | Reverse | CAAGGTTGGTGTCCCTGG | 22 |
| iNOS | Forward | ACCTTGTTCAGCTACGCCTT | 23 |
| iNOS | Reverse | CATTCCCAAATGTGCTTGTC | 24 |
| CD86 | Forward | ACGATGGACCCCAGATGCACCA | 25 |
| CD86 | Reverse | GCGTCTCCACGGAAACAGCA | 26 |
| Arginase 1 | Forward | TTAGGCCAAGGTGCTTGCTGCC | 27 |
| Arginase 1 | Reverse | TACCATGGCCCTGAGGAGGTTC | 28 |
| IFN-α | Forward | AAGGACAGGAAGGATTTTGGATT | 29 |
| IFN-α | Reverse | GAGCCTTCTGGATCTGTTGGTT | 30 |
| IL-6 | Forward | GAGGATACCACTCCCAACAGACC | 31 |
| IL-6 | Reverse | AAGTGCATCATCGTTGTTCATACA | 32 |
| IL-1ß | Forward | CCAAAAGATGAAGGGCTGCTT | 33 |
| IL-1ß | Reverse | GAAAAGAAGGTGCTCATGTCCTC | 34 |
| TNF-α | Forward | TCTCATTCCTGCTTGTGGCA | 35 |
| TNF-α | Reverse | GGTGGTTTGCTACGACGTGG | 36 |
| Bax | Forward | AGCAAACTGGTGCTCAAGGC | 37 |
| Bax | Reverse | CCACAAAGATGGTCACTGTC | 38 |
| Bcl2 | Forward | GTGGTGGAGGAACTCTTCAG | 39 |
| Bcl2 | Reverse | GTTCCACAAAGGCATCCCAG | 40 |
| Caspase-3 | Forward | CCTCAGAGAGACATTCATGG | 41 |
| Caspase-3 | Reverse | GCAGTAGTCGCCTCTGAAGA | 42 |

### <Example 12> Protein Extraction and Western Blot Analysis

Western blot analysis was performed using myoblasts treated with halofuginone after stimulation with TGF-β1 in <Example 1>, fibroblasts treated with halofuginone separated from ALS animal models in <Example 5>, or lumbar spinal cord or knee joints separated after administration of halofuginone to ALS animal models as in <Example 6>.

Specifically, the lumbar spinal cord or knee joint of the mouse of Example 6 was separated and immediately frozen at −80° C. Each lumbar spinal cord or knee joint was then homogenized and chemically treated to obtain cells. The chemically treated cells, the C2C12 cells of Example 1 or the fibroblasts of Example 5 were dissolved in a RIPA buffer (Thermo, MA, USA) with a protease inhibitor and a phosphatase inhibitor (Roche, IN, USA) and incubated on ice for 30 minutes. After centrifugation of the lysate at 13,000 rpm at 4° C. for 20 min, insoluble substances were removed. Protein concentrations were measured by BCA analysis (Pierce Biotechnology). Equal volumes of cell lysate were loaded onto SDS-PAGE gels for electrophoresis, and transferred onto nitrocellulose membranes (Amersham Protran 0.2 um NC, Amersham Pharmacia Biotech, Piscataway, NJ, USA). The membranes were then blocked with 5% (w/v) skimmilk in 1×TBST for 1 h at room temperature. After blocking, anti-RB-actin antibody (1:5000; Santa Cruz, cat #sc47778), anti-Smad2 antibody (1:1000; Cell Signaling, cat #5339), anti-p-Smad2 antibody (1:1000; Cell Signaling, cat #3108), anti-TGF-β1 antibody (1:1000; Santa Cruz, cat #sc130348), anti-α-SMA antibody (1:10000; abcam cat #ab7817), anti-MyoD antibody (1:1000; cat #sc377460), anti-collagen|antibody (1:1000; abcam cat #ab21286), anti-cleaved caspase-3 antibody (1:1000; Cell Signaling, cat #9661), anti-bax antibody (1:500; Santa Cruz, cat #sc493), anti-bcl2 antibody (1:1000; Novus Biologics, cat #NB100-56098), anti-ChAT antibody (1:1000; Millipore, cat #AB144) were treated and incubated overnight at 4° C. Subsequently, membranes were washed several times with 1×TBST and incubated with horseradish peroxidase-conjugated secondary antibodies (anti-mouse, rabbit or goat, 1:5000; GE Healthcare) in blocking buffer for 1 h at room temperature (RT). The membranes were then washed, briefly incubated with the SuperSignal West Pico Plus Chemical Substructure (Pierce Biotechnology) according to the manufacturer's procedure, and quantified with an image analyzer (Amersham Pharmacia Biotech, Piscataway, NJ, USA). β-actin was used as an internal control group. The concentration measurement of protein intensity was quantified using Image J (National Institutes of Health).

### <Example 13> Statistical Analysis

All data were expressed as the mean and the standard error of the mean (SEM) based on at least three independent experiments and as the ratio of control values. The significance of differences between groups was analyzed by Student's t-test or one-way ANOVA followed by Tukey's post host comparison. A p-value of <0.05 was considered statistically significant. The cumulative probability of symptom onset, rotarod failure, and disease endpoint was analyzed using a Kaplan Meier curve. All analyses were performed using GraphPad Prism.

### <Experimental Example 1> Confirmation of Fibrosis Enhancement and Muscle Formation Suppression by TGF-β1 Stimulation in Myoblasts

To investigate the effect of TGF-β1 on myoblasts, real-time qRT-PCR analysis and Western blot analysis were performed after TGF-β1 stimulation on C2C12 mouse myoblasts cell lines.

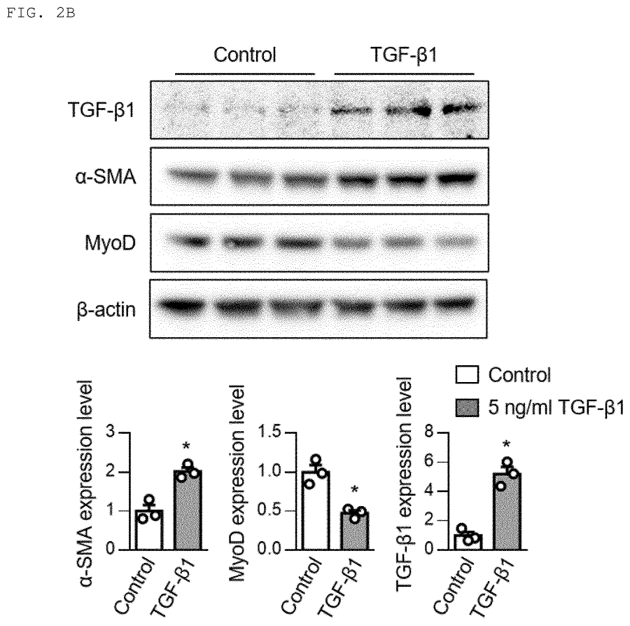

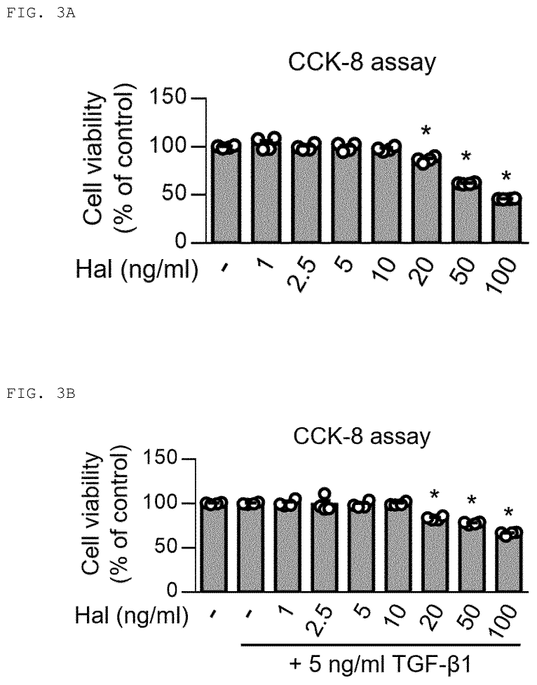

Specifically, after treating C2C12 cells with TGF-β1 for 24 hours as in Example 1, real-time qRT-PCR was performed in the same manner as in Example 11 to confirm TGF-β1, 2 and 3, α-SMA and MyoD mRNA expression (FIG. 2a), and Western blot analysis was performed in the same manner as in Example 12 to confirm autocrine expression of TGF-β1 and MyoD protein and α-SMA (FIG. 2b).

As a result, as shown in FIG. 2a, after 24 hours of TGF-β1 stimulation on the C2C12 cell line, mRNA levels of TGF-β1, 2 and 3 were significantly increased as compared to the control group. Also, it was confirmed that the activation of myoblasts to myofibroblasts by TGF-β stimulation was significantly increased as the mRNA expression of α-SMA. In contrast, the mRNA level of MyoD was significantly decreased after TGF-β stimulation (FIG. 2a).

Also, as shown in FIG. 2b, after 24 hours of TGF-β1 stimulation on the C2C12 cell line, TGF-β1 and α-SMA protein expression were significantly increased. In contrast, protein expression of MyoD was significantly decreased after TGF-β stimulation (FIG. 2b).

From the above results, it can be seen that fibrosis enhancement and muscle formation suppression by TGF-β1 stimulation are induced in myoblasts.

### <Experimental Example 2> Confirmation of Cytotoxicity of Halofuginone

To investigate the cytotoxicity of halofuginone in myoblasts, cell viability analysis and western blot analysis were performed.

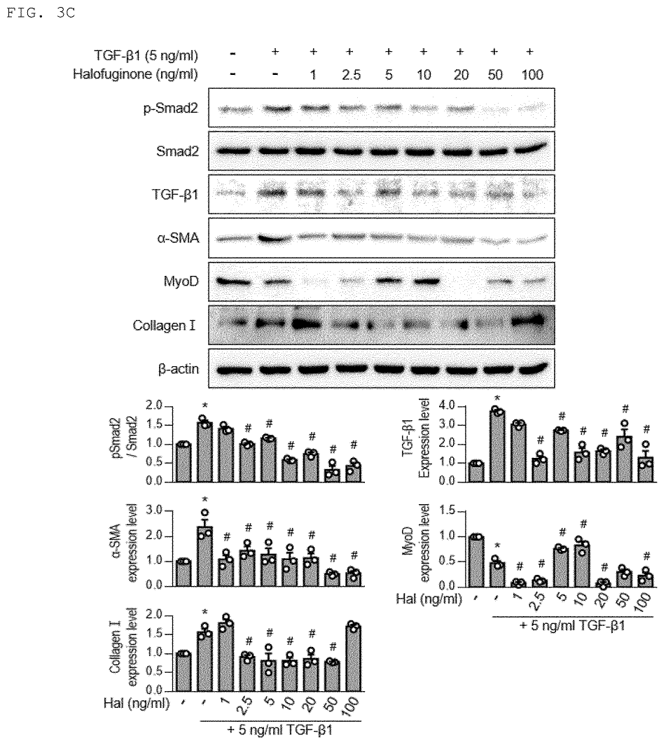

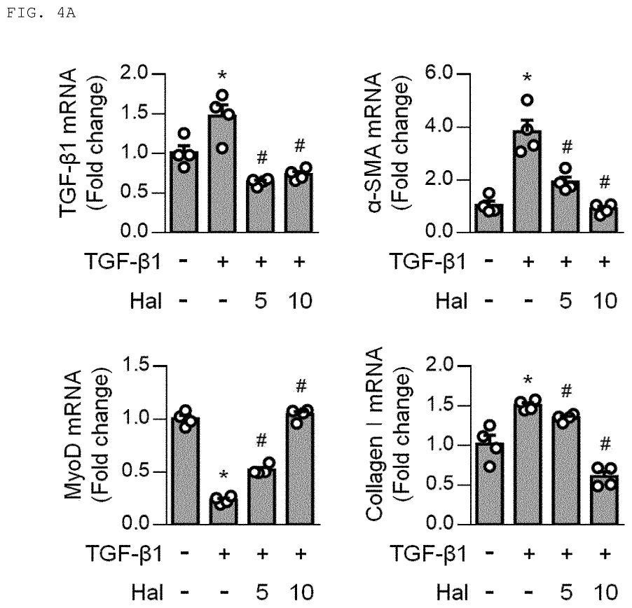

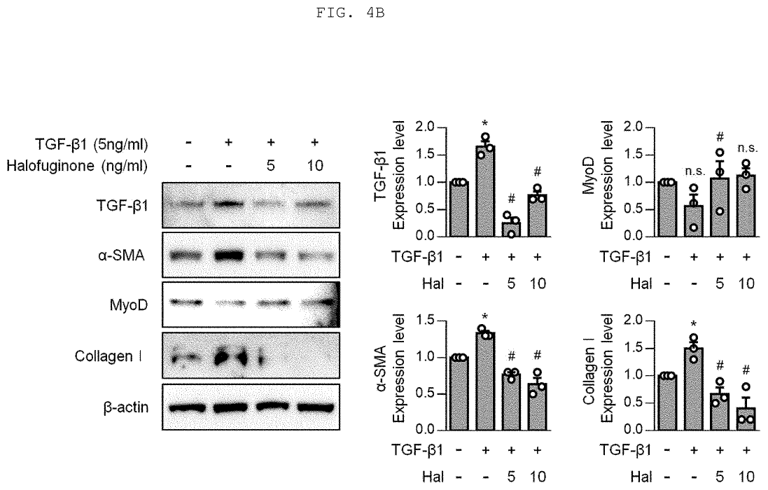

Specifically, C2C12 cells were treated with halofuginone after TGF-β1 stimulation or no stimulation in the same manner as described in Example 2, and cell viability analysis was performed through CCK-8 analysis (FIGS. 3a and 3b), and Western blot analysis was performed in the same manner as described in Example 12 to confirm autocrine expression of p-Smad2, Smad2, TGF-β1, MyoD, collagen I protein and α-SMA (FIG. 3c).

As a result, as shown in FIGS. 3A and 3B, it was confirmed that cell viability was not significantly changed at halofuginone concentration of 1 to 10 ng/ml (FIGS. 3a and 3b).

In addition, as shown in FIG. 3c, at halofuginone concentrations of 5 ng/ml and 10 ng/ml, the increase in p-Smad2/Smad2 ratio, TGF-β1, α-SMA, and collagen I protein levels by TGF-β1 stimulation was significantly reduced, and the decrease in MyoD protein levels by TGF-β1 stimulation was significantly recovered (FIG. 3c).

Therefore, treatment with the optimum concentration of halofuginone in C2C12 cells was determined with the low concentration of 5 ng/ml and the high concentration of 10 ng/ml.

### <Experimental Example 3> Confirmation of Alleviation of Fibrosis Enhancement and Muscle Formation Suppression by Halofuginone in TGF-β1-Stimulated Myoblasts

To investigate whether changes by TGF-β1 in myoblasts could be inhibited by halofuginone as a TGF-β inhibitor, C2C12 mouse myoblasts cell lines were treated with halofuginone after TGF-β1 stimulation, and real-time qRT-PCR analysis, Western blot analysis, and immunocytochemical analysis were performed.

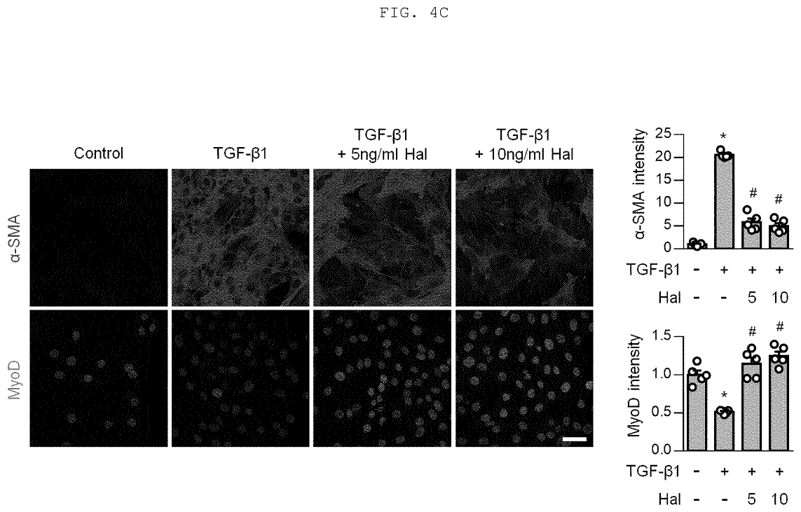

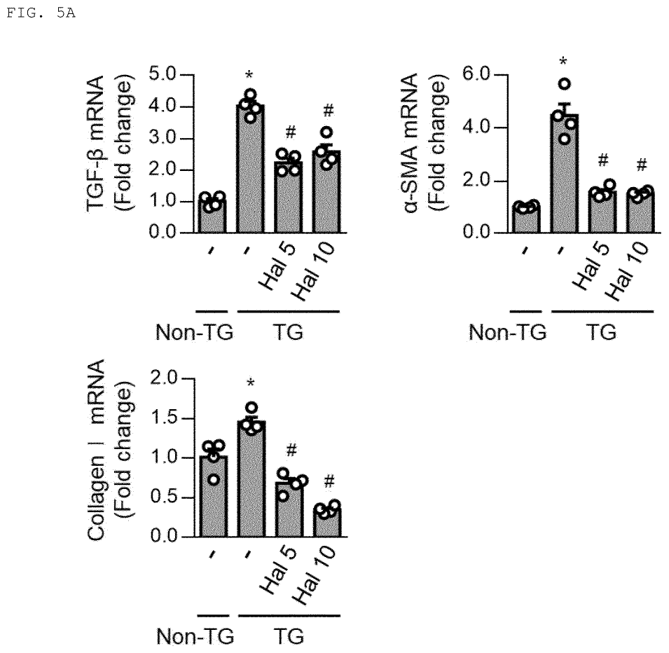

Specifically, as in Example 1, C2C12 cells were stimulated with TGF-β1 for 24 hours, and then halofuginone was treated for 24 hours, and real-time qRT-PCR was performed in the same manner as in Example 11 to confirm TGF-β1, α-SMA, MyoD, and collagen I (Col-I) mRNA expression (FIG. 4a). In addition, Western blot analysis was performed in the same manner as in Example 12 to confirm TGF-β1, α-SMA, MyoD, and collagen I protein expression (FIG. 4b). In addition, immunocytochemical analysis was performed in the same manner as in Example 3 to confirm α-SMA and MyoD expression (FIG. 4c).

As a result, as shown in FIG. 4a and FIG. 4b, in the group treated with low or high concentration halofuginone, an increase in TGF-β1, α-SMA, and collagen I mRNA levels by TGF-β1 stimulation was significantly reduced, and a decrease in MyoD mRNA levels by TGF-β1 stimulation was significantly recovered (FIG. 4a). Protein expression was also the same (FIG. 4b).

In addition, as shown in FIG. 4c, the intensity of α-SMA reduction and MyoD recovery were confirmed by halofuginone low or high concentration treatment using immunofluorescence staining and confocal microscopy (FIG. 4c).

From the above results, it can be seen that fibrosis enhancement and muscle formation suppression by TGF-β1 stimulation in myoblasts can be prevented by using halofuginone as a TGF-β inhibitor.

### <Experimental Example 4> Confirmation of Inhibition of Fibrosis by Halofuginone in Fibroblasts Derived from ALS Animal Models

To investigate the changes in TGF-β1 and the effects of halofuginone in ALS animal model-derived fibroblasts, real-time qRT-PCR analysis and Western blot analysis were performed after treating halofuginone in ALS animal model-derived fibroblasts.

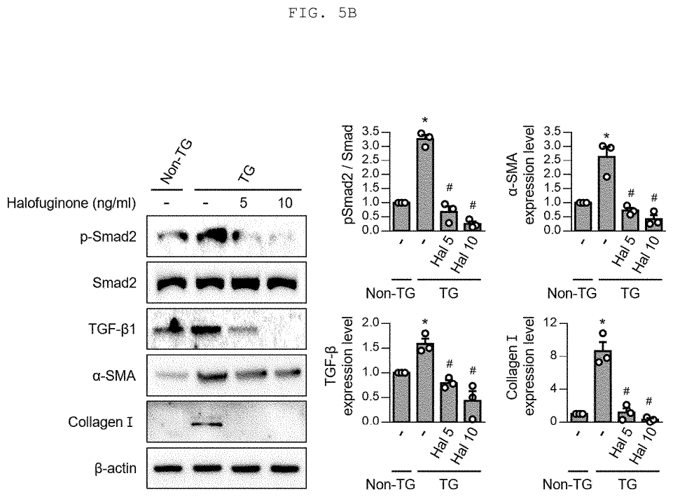

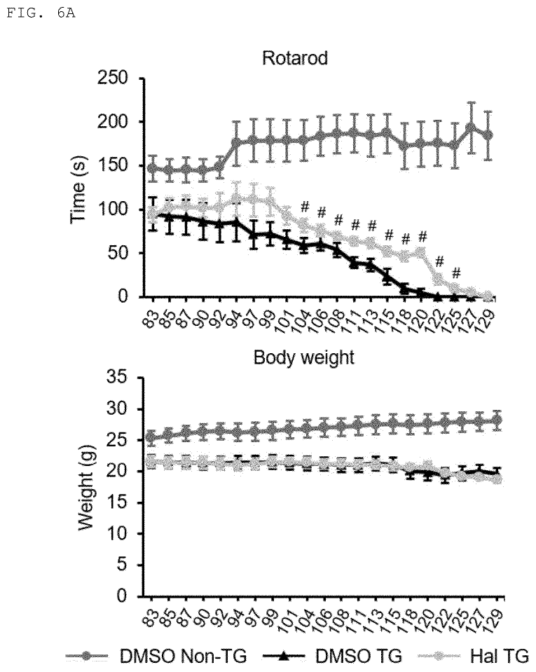

Specifically, after fibroblasts were isolated from an ALS animal model by the same method as described in Example 5, halofuginone was treated, and then real-time qRT-PCR was performed by the same method as described in Example 11 to confirm TGF-β1, α-SMA, and collagen I (Col-I) mRNA expression (FIG. 5a). In addition, Western blot analysis was performed by the same method as described in Example 12 to confirm p-Smad2, Smad2, TGF-β1, α-SMA and collagen I protein expression (FIG. 5b). As a control group, fibroblasts isolated from non-transgenic mice were used.

As a result, as shown in FIG. 5a and FIG. 5b, the p-Smad2/Smad2 ratio and the protein expression of TGF-β, α-SMA, and Col-I as well as the level of mRNA were significantly elevated in G93A mutant SOD1 mouse-derived fibroblasts compared to non-transformed mouse-derived fibroblasts (Non-TG) (FIG. 5A and FIG. 5B). It was confirmed that this change was attenuated by treatment with low or high concentration halofuginone for 24 hours (FIG. 5a and FIG. 5b).

From the above results, it can be seen that fibrosis is enhanced in fibroblasts of the ALS mouse model, and fibrosis can be suppressed by halofuginone treatment.

### <Experimental Example 5> Confirmation of Delayed Onset of ALS and Improved Performance by Halofuginone Administration in ALS Animal Models

To evaluate the effectiveness of halofuginone in vivo, halofuginone was administered to ALS animal models and disease progression, survival, and motor function were analyzed.

Specifically, halofuginone was administered to the ALS animal model in the same manner as described in <Example 6>, and disease progression, survival, and motion function analysis were performed in the same manner as described in <Example 7>.

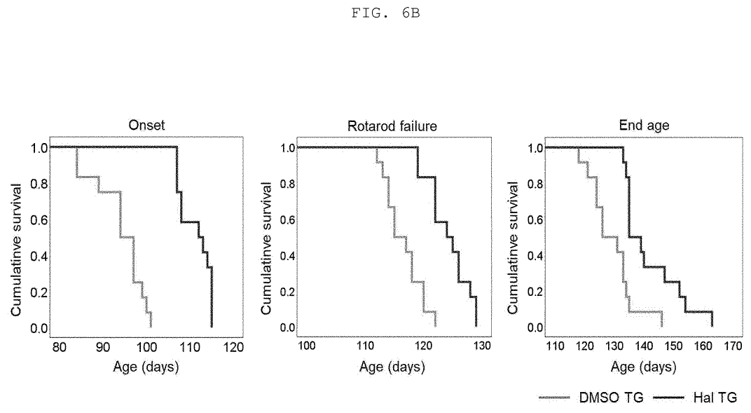

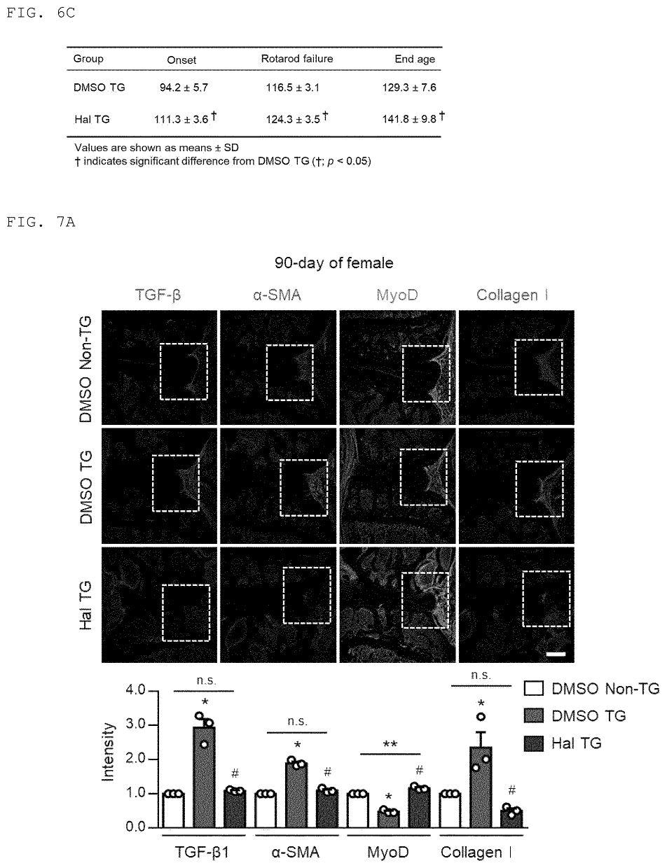

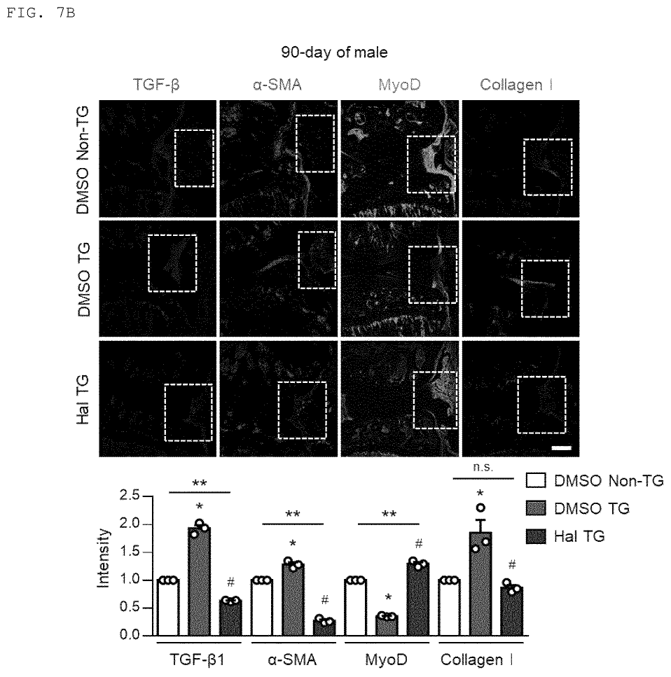

As a result, as shown in FIG. 6a, each TG group in the symptom stage showed a significantly shorter time in the rotarod than in the non-TG group. In the TG group, the halofuginone-administered TG group (Hal TG group) showed a significantly longer time in the rotarod compared to the DMSO-administered TG group (DMSO TG group). The body weight was significantly reduced in the TG group compared to the non-TG group, and there was no significant difference between the DMSO TG group and the Hal TG group (FIG. 6a).

In addition, as shown in FIGS. 6B and 6C, there was a difference in the onset of symptoms between the DMSO TG group and the Hal TG group, and the onset of symptoms in the Hal TG group was significantly delayed compared to the DMSO TG group (mean±standard deviation [SD], 113±3.6 vs 94.2±5.7, p<0.5 respectively). In addition, the age of reaching rotarod failure was delayed in the Hal TG group than in the DMSO TG group (124.3±3.5 vs 116.5±3.1, p<0.001, respectively), and the endpoint age was also significantly increased (141.8±9.8 vs 129.3±7.6, FIGS. 6b and 6c), respectively).

### <Experimental Example 6> Confirmation of Decreased Fibrosis of Synovial Cavity and Improved Skeletal Muscle Production by Halofuginone Administration in the Early Stages of Disease in ALS Animal Model

To evaluate the effect of halofuginone on the onset of ALS, halofuginone was administered to an ALS animal model, and the changes in the synovial cavity of the knee joint were observed at the early and late stages of the disease through immunohistochemical analysis (IHC).

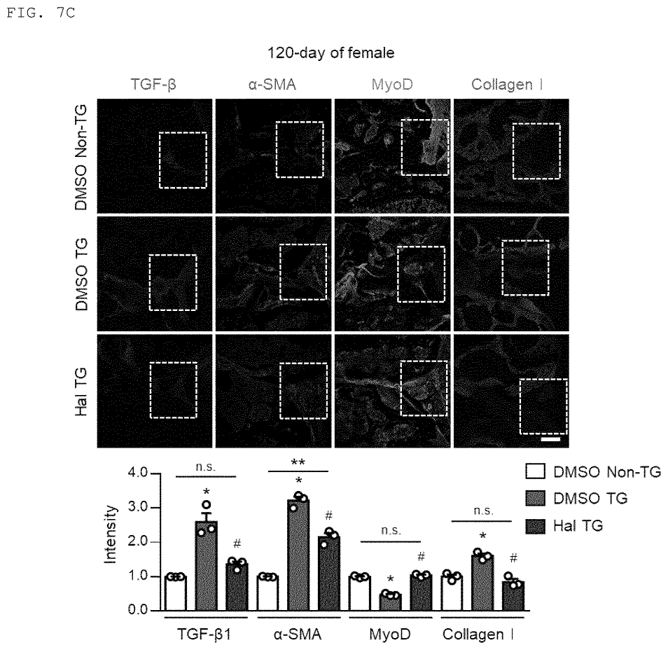

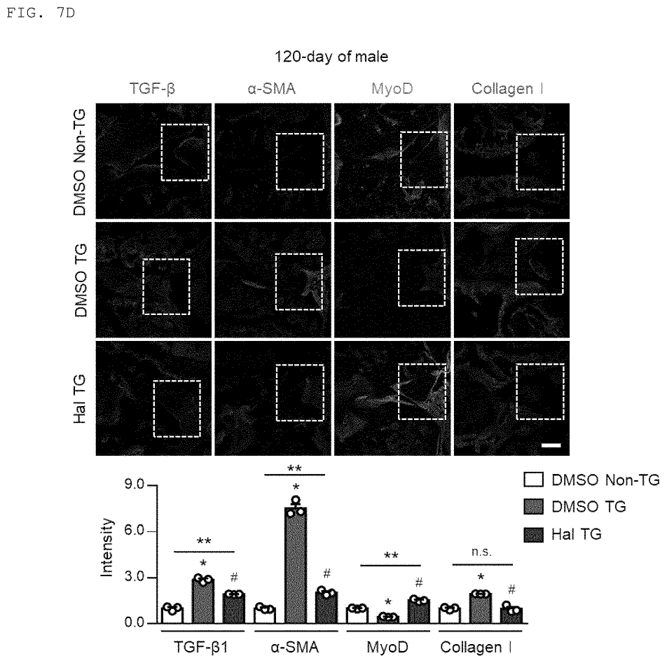

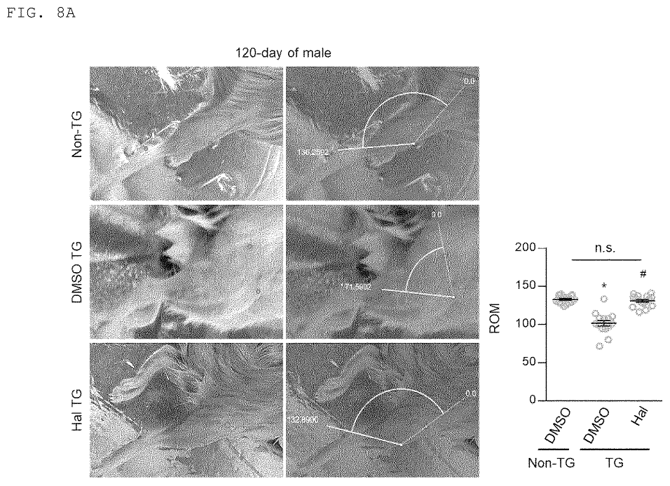

Specifically, halofuginone was administered to G93A mutant SOD1 mice from the early onset of ALS (day 73) in the same manner as described in Example 6. Immunohistochemical analysis was performed on the knee joints of early-stage female and male mice (90 days old, FIGS. 7a and 7b) and late-stage female and male mice (120 days old, FIGS. 7c and 7d) in the same manner as described in <Example 8>.

As a result, as shown in FIG. 7a and FIG. 7b, compared to the DMSO Non-TG group, the DMSO TG group confirmed a significant increase in TGF-β1 in the knee joint synovial cavity in the early stages of the disease regardless of gender. In addition, it was confirmed that α-SMA and Col-I increased and MyoD decreased with an increase in TGF-β1 in the synovial cavity of the DMSOTG group compared to the DMSO Non-TG group. On the other hand, in the Hal TG group, the increase in TGF-β was suppressed in the early stages of the disease, and accordingly, α-SMA and Col-I expression were also significantly lower than in the DMSO TG group (FIGS. 7a and 7b).

In addition, as shown in FIG. 7c and FIG. 7d, in the Hal TG group, significantly lower TGF-β1, α-SMA, Col-I, and higher MyoD were consistently maintained in the synovial cavity compared to the DMSO TG group. In addition, the strength of TGF-β1, MyoD, and Col-1 in the female Hal TG group was not different from that of the DMSO non-TG group, and the strength of MyoD in the male Hal TG group was slightly increased compared to the DMSO non-TG group (FIGS. 7c and 7d).

From the above results, it can be seen that myofibroblast activation and enhanced fibrosis in the synovial cavity of ALS mice due to an increase in TGF-β can be improved by administration of halofuginone. Furthermore, it can be observed that the reduction in myogenesis of skeletal muscle in the synovial cavity of ALS mice can be prevented by the administration of halofuginone. Moreover, it can be observed that the administration of halofuginone at the early stages of the disease in ALS mice significantly reduces fibrosis in the synovial cavity and enhances muscle formation, irrespective of gender.

### <Experimental Example 7> Confirmation of Improvement in Joint Contracture in ALS Animal Model by Halofuginone Administration

To investigate the functional resistance due to fibrosis in ALS and the effect of halofuginone on it, range of motion (ROM) of the knee joint was measured, and Western blot analysis was performed after administering halofuginone to an ALS animal model.

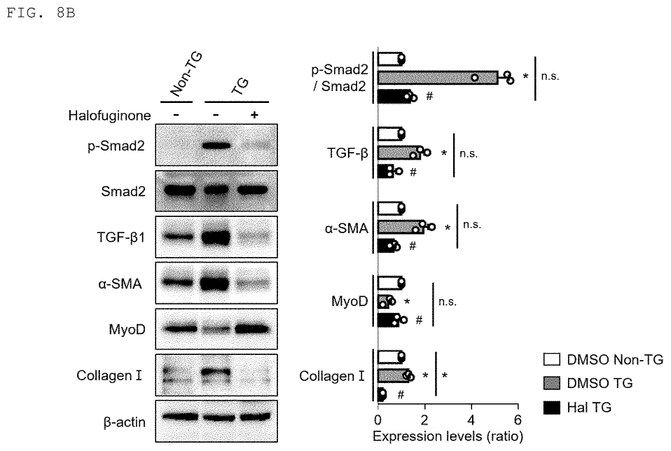

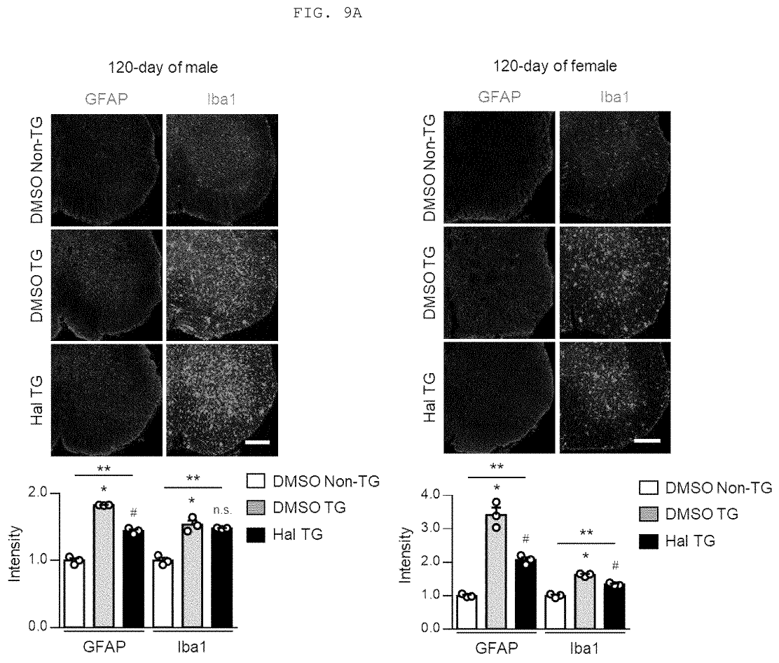

Specifically, halofuginone was administered to G93A mutant SOD1 mice in the same manner as described in Example 6, and ROM measurement of the knee joint was performed in the same manner as described in Example 10 (FIG. 8a). In addition, Western blot analysis was performed in the same manner as the method described in Example 12 using knee joint tissue to confirm p-Smad2, Smad2, TGF-β1, α-SMA, MyoD, and collagen I protein expression (FIG. 8b).

As a result, as shown in FIG. 8A, it was confirmed that ROM was significantly reduced in the DMSO TG group compared to the 120-day-old DMSO Non-TG group, and ROM was not changed in the Hal TG group (FIG. 8a).

In addition, as shown in FIG. 8b, significant increases in p-Smad2/Smad2 ratio, TGF-β, α-SMA, and Col-I and decreases in MyoD were confirmed in the DMSO TG group compared to the DMSO Non-TG group. In addition, the p-Smad2/Smad2 ratio of the Hal TG group and the expression levels of TGF-β1, α-SMA, and MyoD were preserved similarly to DMSO non-TG mice, and Col-1 expression levels were rather reduced (FIG. 8b).

Through the above results, it can be observed that the increase of TGF-β in ALS mice enhances fibrosis within the knee joint, leading to joint contracture, while administration of halofuginone can improve this condition.

### <Experimental Example 8> Confirmation of Anti-Inflammatory and Neuroprotective Effects of Halofuginone Administration in CNS Tissues of ALS Animal Model

To investigate the effects of halofuginone administration on the central nervous system (CNS) in ALS, immunohistochemical analysis, motor neuron cell counting in the lumbar spinal cord, and Western blot analysis were performed using the lumbar spinal cord after administering halofuginone to an ALS animal model.

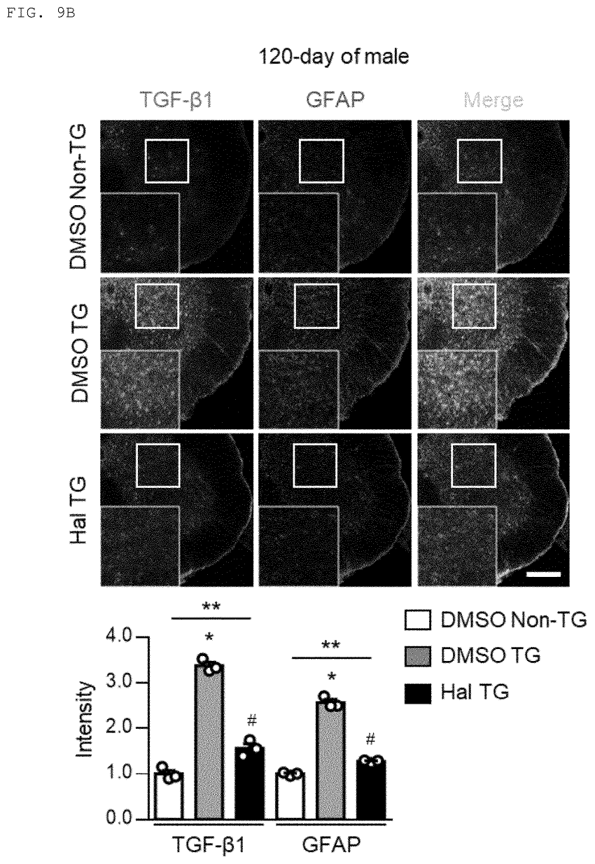

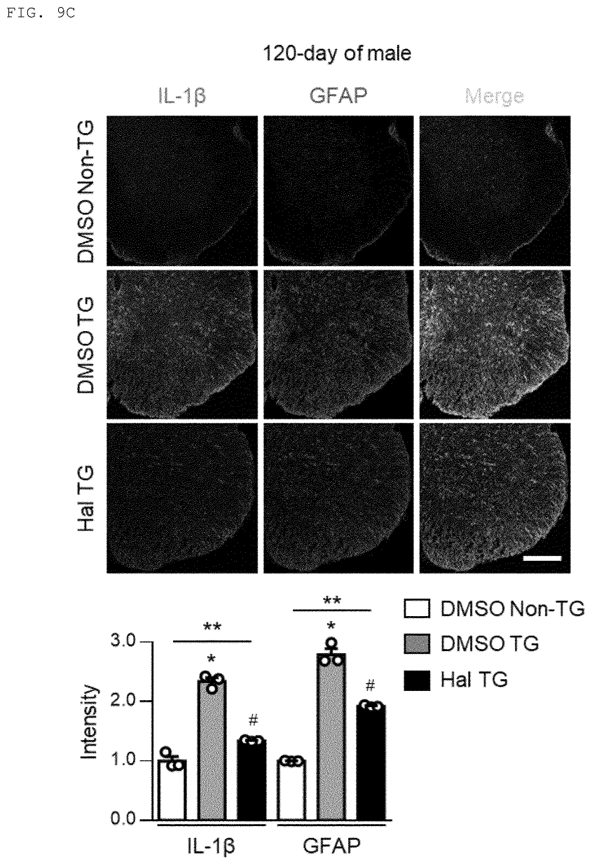

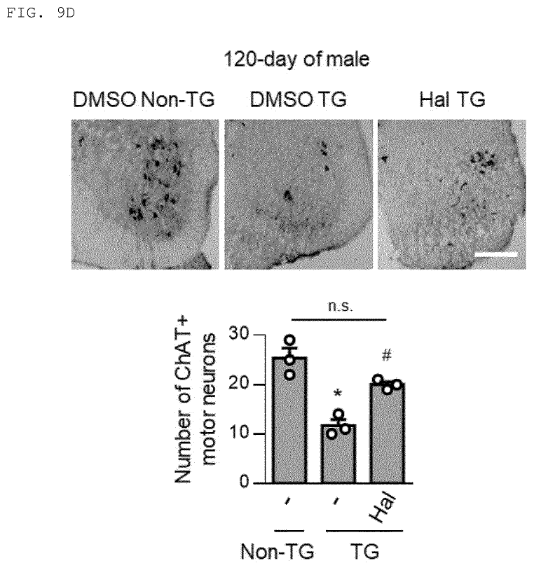

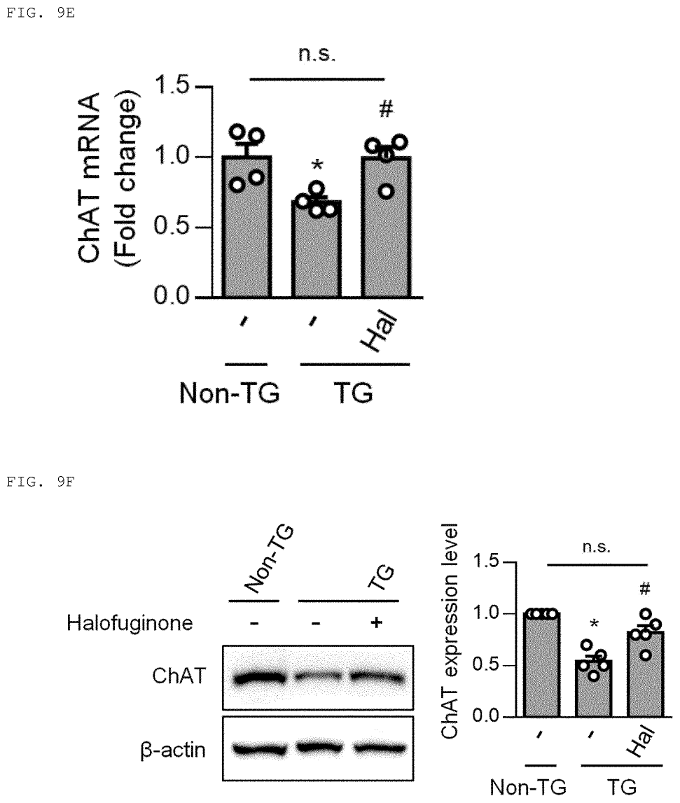

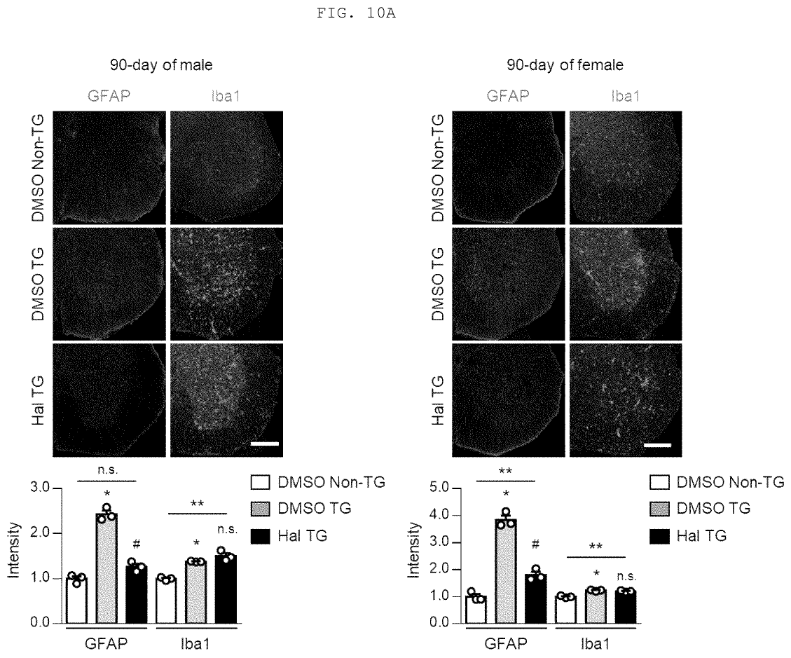

Specifically, halofuginone was administered to G93A mutant SOD1 mice in the same manner as described in <Example 6>, and immunohistochemical analysis was performed in the same manner as described in <Example 8> to identify TGF-β1 and glial cells in the lumbar spinal cord. Considering that TGF-β derived from astrocytes has been reported to accelerate disease in ALS mice, each group's spinal cord was co-stained with TGF-β and GFAP during immunohistochemical analysis. In addition, Iba1 staining was performed to confirm the activity of microglia (FIGS. 9a and 9b).

In addition, to investigate the effect of glial changes in TGF-β and CNS on inflammation, IL-1β, a representative pro-inflammatory cytokine, was simultaneously stained with GFAP during immunohistochemical analysis (FIG. 9c).

In addition, to investigate the motor neuron source of the spinal cord, motor neurons of the lumbar spinal cord were observed and counted in the same manner as described in Example 9 (FIG. 9d), and real-time qRT-PCR and Western blot analysis were performed in the same manner as described in Example 11 and Example 12 to quantitatively analyze the expression of ChAT mRNA (FIG. 9e) and ChAT protein (FIG. 9f).

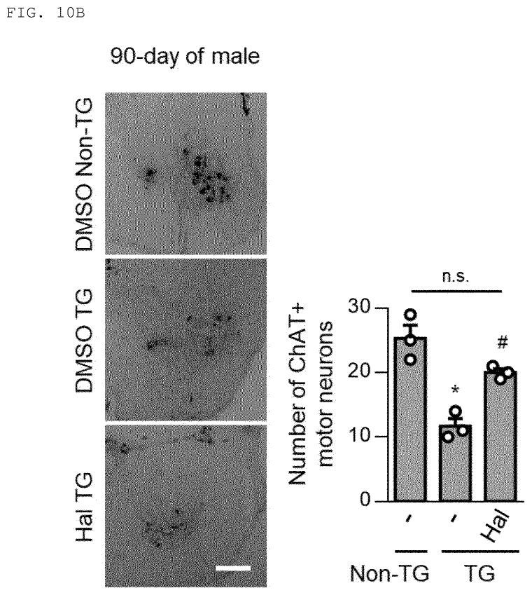

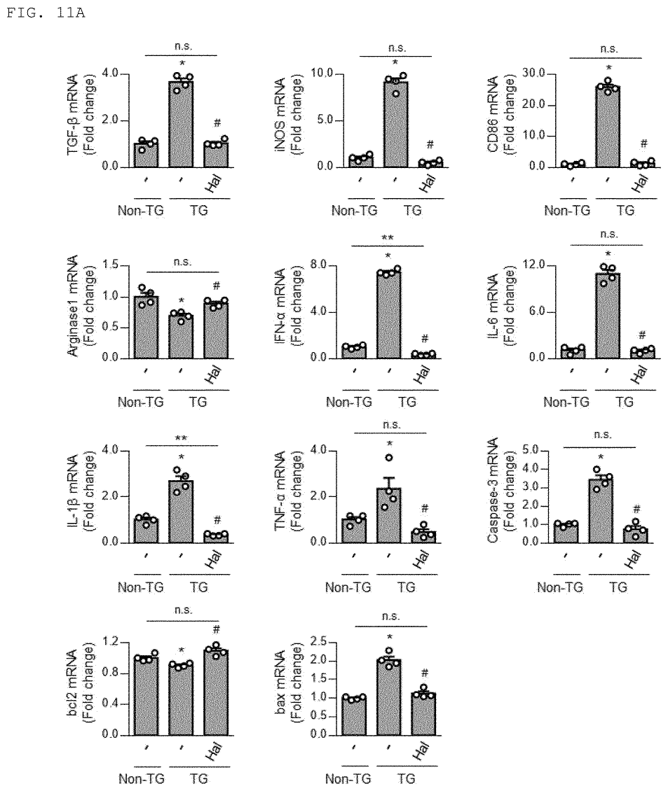

In addition, halofuginone was administered to G93A mutant SOD1 mice in the same manner as described in Example 6. After sacrificing the mice at 90 days of age, Iba1 staining was performed to confirm the activation of microglia (FIG. 10a), and motor neurons were observed and counted using the same method as described above (FIG. 10b).

As a result, as shown in FIGS. 9A and 9B, GFAP strength was significantly increased in the DMSO TG group of 120-day-old mice compared to the DMSO Non-TG group, and TGF-β1 was also increased at the same site. It was confirmed that such an increase in astrocyte activity and TGF-β1 was significantly reduced in the Hal TG group (FIG. 9b). Meanwhile, it was confirmed that microglia were significantly increased in the DMSO TG group, and microglia were continuously increased in the Hal TG group (FIG. 9a)

In addition, as shown in FIG. 9c, compared to the DMSO non-TG group of 120-day-old mice, IL-1β increased with GFAP activity in the DMSO TG group, and this activated inflammation was suppressed in the Hal TG group (FIG. 9c).

In addition, as shown in FIGS. 9D to 9F, all mRNA levels of ChAT, the number of ChAT-positive motor neurons, and ChAT expression in the 120-day-old mouse DMSO TG group were significantly lower than in the DMSO Non-TG group, whereas in the Hal TG group, it was confirmed that they were preserved similarly to the DMSO Non-TG group (FIGS. 9d to 9f).

In addition, as shown in FIGS. 10a and 10b, the above changes were similar in 90-day-old mice (FIGS. 10a and 10b).

From the above results, it can be seen that an increase in TGF-β in the CNS of ALS mice is associated with an increase in the inflammatory response and a decrease in motor neurons accordingly, and this process can be suppressed by halofuginone administration.

### <Experimental Example 9> Confirmation of Chronic Inflammation and Inhibition of Neuronal Cell Death in CNS by Halofuginone Administration in ALS Animal Model

To investigate the effect of halofuginone administration on the inflammatory response of CNS in ALS, real-time qRT-PCR analysis and western blot analysis were performed using lumbar spinal cord after administration of halofuginone in ALS animal models.

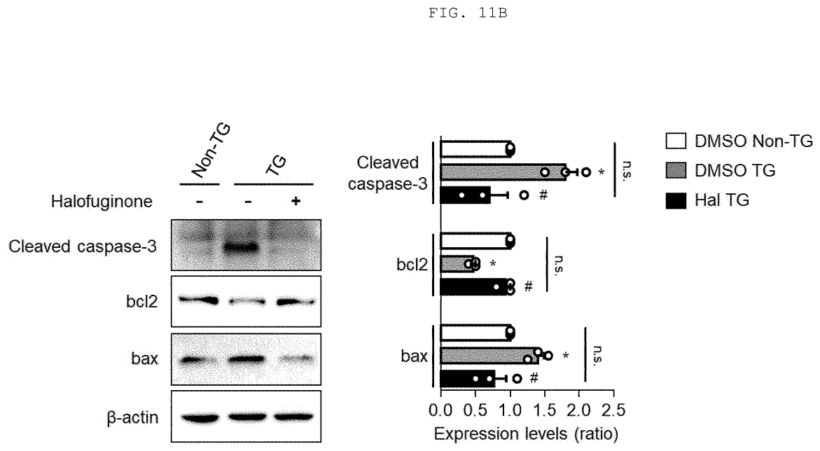

Specifically, Halofuginone was administered to G93A mutant SOD1 mice in the same manner as described in <Example 6>, and lumbar spinal cord was isolated after 120 days of age of mice were sacrificed. Then, mRNA expression of inflammation-related factors and neuronal cell death-related factors of CNS was confirmed by performing real-time qRT-PCT analysis in the same manner as in Example 11 (FIG. 11a), and protein expression of neuronal cell death-related factors was confirmed by performing Western blot analysis in the same manner as in Example 12 (FIG. 11b). Since the role (inflammatory or anti-inflammatory effect) differs according to the M1 or M2 subtype of microglia, the expression of M1 marker (iNOS, CD86), M2 marker (arginase 1) and pro-inflammatory factors (IFN-a, TNF-a, IL-1b, and IL-6) were confirmed as factors related to inflammation of CNS. In addition, the expression of caspase-3, bax, and bcl-2 was confirmed as factors related to neuronal apoptosis.

As a result, as shown in FIG. 11a, mRNA levels of M1 markers and proinflammatory factors were significantly increased according to mRNA levels of TGF-β in the DMSO TG group, which was inhibited by halofuginone. In addition, the mRNA level of bcl-2, an anti-neuronal cell death factor, decreased in the DMSO TG group compared to the DMSO Non-TG group, and it was confirmed that it is preserved in the Hal TG group (FIG. 11a).

In addition, as shown in FIG. 11b, compared to the DMSO Non-TG group, the protein expression of cleaved caspase-3 and bax, markers of neuronal cell death, increased, while bcl-2, an anti-neuronal cell death factor, decreased in the DMSO TG group. These changes were observed to be improved in the HAL TG group (FIG. 11b).

Based on the above results, it can be observed that halofuginone blocks sustained increase of TGF-β in ALS mice, thereby exhibiting anti-inflammatory effects in the CNS and suppressing neuronal cell death.

Through the results from <Experimental Example 1> to <Experimental Example 9>, it has been confirmed that halofuginone exhibits dual therapeutic effects in ALS: improvement of joint contracture due to increased TGF-β and suppression of chronic inflammation and neuronal cell death in the CNS. Therefore, halofuginone could be utilized for the prevention or treatment of neurodegenerative or motor neuron diseases, including ALS, associated with increased TGF-β levels.

### INDUSTRIAL APPLICABILITY

In the present invention, it was confirmed that in ALS cell and animal models, halofuginone inhibits fibrosis induced by elevated TGF-β, enhances skeletal muscle formation, improves joint contracture, and exhibits dual effects of suppressing inflammatory responses and neuronal cell death in the central nervous system, and then, halofuginone leads to the delay in the progression of ALS symptoms, improvement in performance capabilities, and extension of survival duration. Consequently, halofuginone or pharmaceutically acceptable salts thereof can be usefully utilized as an active ingredient in a composition for the prevention or treatment of neurodegenerative or motor neuron diseases including ALS.

1. A method for preventing or treating neurodegenerative or motor neuron diseases, comprising the step of administering to a subject halofuginone or a pharmaceutically acceptable salt thereof.

2. The method according to claim 1 for preventing or treating neurodegenerative or motor neuron diseases, wherein the halofuginone or a pharmaceutically acceptable salt thereof is a TGF-β inhibitor.

3. The method according to claim 1 for preventing or treating neurodegenerative or motor neuron diseases, wherein the halofuginone is a compound represented by the following [Chemical Formula 1]:

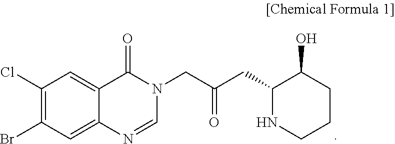 [SMILES: O=C(C[C@H]1NCCC[C@@H]1O)Cn1cnc2cc(Br)c(Cl)cc2c1=O]

4. The method according to claim 1 for preventing or treating neurodegenerative or motor neuron diseases, wherein the halofuginone alleviates fibrosis of the joint synovial cavity and enhances skeletal muscle generation.

5. The method according to claim 1 for preventing or treating neurodegenerative or motor neuron diseases, wherein the halofuginone inhibits inflammatory responses and neuronal cell death in the central nervous system.

6. The method according to claim 1 for preventing or treating neurodegenerative or motor neuron diseases, wherein the halofuginone delays the progression of symptoms of neurodegenerative or motor neuron diseases, improves performance capabilities and extends survival duration.

7. The method according to claim 1 for preventing or treating neurodegenerative or motor neuron diseases, wherein the neurodegenerative or motor neuron disease is amyotrophic lateral sclerosis, Alzheimer's disease, Parkinson's disease, Huntington's disease, multiple sclerosis, dystonia, spinal muscular atrophy, or inflammatory neuropathy.


SEQUENCE LISTING (42 sequences)

SEQ ID NO: 1 - DNA, 24 bp, synthetic construct
  misc_feature 1..24: note = hSOD1wt primer_forward
  source 1..24: mol_type = other DNA
ctaggccacagaattgaaagatct

SEQ ID NO: 2 - DNA, 25 bp, synthetic construct
  misc_feature 1..25: note = hSOD1wt primer_reverse
  source 1..25: mol_type = other DNA
gtaggtggaaattctagcatcatcc

SEQ ID NO: 3 - DNA, 21 bp, synthetic construct
  misc_feature 1..21: note = hSOD1G93A primer_forward
  source 1..21: mol_type = other DNA
catcagccctaatccatctga

SEQ ID NO: 4 - DNA, 21 bp, synthetic construct
  misc_feature 1..21: note = hSOD1G93A primer_reverse
  source 1..21: mol_type = other DNA
cgcgactaacaatcaaagtga

SEQ ID NO: 5 - DNA, 20 bp, synthetic construct
  misc_feature 1..20: note = GAPDH primer_forward
  source 1..20: mol_type = other DNA
tgtgtccgtcgtggatctga

SEQ ID NO: 6 - DNA, 21 bp, synthetic construct
  misc_feature 1..21: note = GAPDH primer_reverse
  source 1..21: mol_type = other DNA
cctgcttcaccaccttcttga

SEQ ID NO: 7 - DNA, 19 bp, synthetic construct
  misc_feature 1..19: note = TGF-beta primer_forward
  source 1..19: mol_type = other DNA
caccggagagccctggata

SEQ ID NO: 8 - DNA, 19 bp, synthetic construct
  misc_feature 1..19: note = TGF-beta primer_reverse
  source 1..19: mol_type = other DNA
tgtacagctgccgcacaca

SEQ ID NO: 9 - DNA, 20 bp, synthetic construct
  misc_feature 1..20: note = TGF-beta1 primer_forward
  source 1..20: mol_type = other DNA
tcgctttgtacaacagcacc

SEQ ID NO: 10 - DNA, 20 bp, synthetic construct
  misc_feature 1..20: note = TGF-beta1 primer_reverse
  source 1..20: mol_type = other DNA
actgcttcccgaatgtctga

SEQ ID NO: 11 - DNA, 20 bp, synthetic construct
  misc_feature 1..20: note = TGF-beta2 primer_forward
  source 1..20: mol_type = other DNA
aggcagagttcagggtcttc

SEQ ID NO: 12 - DNA, 20 bp, synthetic construct
  misc_feature 1..20: note = TGF-beta2 primer_reverse
  source 1..20: mol_type = other DNA
ccttgctatcgatgtagcgc

SEQ ID NO: 13 - DNA, 20 bp, synthetic construct
  misc_feature 1..20: note = TGF-beta3 primer_forward
  source 1..20: mol_type = other DNA
aggagacctcggagtctgag

SEQ ID NO: 14 - DNA, 22 bp, synthetic construct
  misc_feature 1..22: note = TGF-beta3 primer_reverse
  source 1..22: mol_type = other DNA
cactgaggacacattgaaacga

SEQ ID NO: 15 - DNA, 25 bp, synthetic construct
  misc_feature 1..25: note = alpha-SMA primer_forward
  source 1..25: mol_type = other DNA
agttttgtgctgaggtccctatatg

SEQ ID NO: 16 - DNA, 21 bp, synthetic construct
  misc_feature 1..21: note = alpha-SMA primer_reverse
  source 1..21: mol_type = other DNA
ttcccaaacaaggagcaaaga

SEQ ID NO: 17 - DNA, 20 bp, synthetic construct
  misc_feature 1..20: note = MyoD primer_forward
  source 1..20: mol_type = other DNA
tacagtggcgactcagatgc

SEQ ID NO: 18 - DNA, 20 bp, synthetic construct
  misc_feature 1..20: note = MyoD primer_reverse
  source 1..20: mol_type = other DNA
gagatgcgctccactatgct

SEQ ID NO: 19 - DNA, 19 bp, synthetic construct
  misc_feature 1..19: note = Collagen I primer_forward
  source 1..19: mol_type = other DNA
ctggcggttcaggtccaat

SEQ ID NO: 20 - DNA, 19 bp, synthetic construct
  misc_feature 1..19: note = Collagen I primer_reverse
  source 1..19: mol_type = other DNA
ttccaggcaatccacgagc

SEQ ID NO: 21 - DNA, 19 bp, synthetic construct
  misc_feature 1..19: note = ChAT primer_forward
  source 1..19: mol_type = other DNA
ctgtgcccccttctagagc

SEQ ID NO: 22 - DNA, 18 bp, synthetic construct
  misc_feature 1..18: note = ChAT primer_reverse
  source 1..18: mol_type = other DNA
caaggttggtgtccctgg

SEQ ID NO: 23 - DNA, 20 bp, synthetic construct
  misc_feature 1..20: note = iNOS primer_forward
  source 1..20: mol_type = other DNA
accttgttcagctacgcctt

SEQ ID NO: 24 - DNA, 20 bp, synthetic construct
  misc_feature 1..20: note = iNOS primer_reverse
  source 1..20: mol_type = other DNA
cattcccaaatgtgcttgtc

SEQ ID NO: 25 - DNA, 22 bp, synthetic construct
  misc_feature 1..22: note = CD86 primer_forward
  source 1..22: mol_type = other DNA
acgatggaccccagatgcacca

SEQ ID NO: 26 - DNA, 20 bp, synthetic construct
  misc_feature 1..20: note = CD86 primer_reverse
  source 1..20: mol_type = other DNA
gcgtctccacggaaacagca

SEQ ID NO: 27 - DNA, 22 bp, synthetic construct
  misc_feature 1..22: note = Arginase 1 primer_forward
  source 1..22: mol_type = other DNA
ttaggccaaggtgcttgctgcc

SEQ ID NO: 28 - DNA, 22 bp, synthetic construct
  misc_feature 1..22: note = Arginase 1 primer_reverse
  source 1..22: mol_type = other DNA
taccatggccctgaggaggttc

SEQ ID NO: 29 - DNA, 23 bp, synthetic construct
  misc_feature 1..23: note = IFN-alpha primer_forward
  source 1..23: mol_type = other DNA
aaggacaggaaggattttggatt

SEQ ID NO: 30 - DNA, 22 bp, synthetic construct
  misc_feature 1..22: note = IFN-alpha primer_reverse
  source 1..22: mol_type = other DNA
gagccttctggatctgttggtt

SEQ ID NO: 31 - DNA, 23 bp, synthetic construct
  misc_feature 1..23: note = IL-6 primer_forward
  source 1..23: mol_type = other DNA
gaggataccactcccaacagacc

SEQ ID NO: 32 - DNA, 24 bp, synthetic construct
  misc_feature 1..24: note = IL-6 primer_reverse
  source 1..24: mol_type = other DNA
aagtgcatcatcgttgttcataca

SEQ ID NO: 33 - DNA, 21 bp, synthetic construct
  misc_feature 1..21: note = IL-1beta primer_forward
  source 1..21: mol_type = other DNA
ccaaaagatgaagggctgctt

SEQ ID NO: 34 - DNA, 23 bp, synthetic construct
  misc_feature 1..23: note = IL-1beta primer_reverse
  source 1..23: mol_type = other DNA
gaaaagaaggtgctcatgtcctc

SEQ ID NO: 35 - DNA, 20 bp, synthetic construct
  misc_feature 1..20: note = TNF-alpha primer_forward
  source 1..20: mol_type = other DNA
tctcattcctgcttgtggca

SEQ ID NO: 36 - DNA, 20 bp, synthetic construct
  misc_feature 1..20: note = TNF-alpha primer_reverse
  source 1..20: mol_type = other DNA
ggtggtttgctacgacgtgg

SEQ ID NO: 37 - DNA, 20 bp, synthetic construct
  misc_feature 1..20: note = Bax primer_forward
  source 1..20: mol_type = other DNA
agcaaactggtgctcaaggc

SEQ ID NO: 38 - DNA, 20 bp, synthetic construct
  misc_feature 1..20: note = Bax primer_reverse
  source 1..20: mol_type = other DNA
ccacaaagatggtcactgtc

SEQ ID NO: 39 - DNA, 20 bp, synthetic construct
  misc_feature 1..20: note = Bcl2 primer_forward
  source 1..20: mol_type = other DNA
gtggtggaggaactcttcag

SEQ ID NO: 40 - DNA, 20 bp, synthetic construct
  misc_feature 1..20: note = Bcl2 primer_reverse
  source 1..20: mol_type = other DNA
gttccacaaaggcatcccag

SEQ ID NO: 41 - DNA, 20 bp, synthetic construct
  misc_feature 1..20: note = Caspase-3 primer_forward
  source 1..20: mol_type = other DNA
cctcagagagacattcatgg

SEQ ID NO: 42 - DNA, 20 bp, synthetic construct
  misc_feature 1..20: note = Caspase-3 primer_reverse
  source 1..20: mol_type = other DNA
gcagtagtcgcctctgaaga